(19) United States
(12) Patent Application Publication
(10) Pub. No.: US 2007/0225713 A1
(43) Pub. Date: Sep. 27, 2007

(54) Systems and methods for posterior dynamic stabilization of the spine

(76) Inventors: Moti Altarac, Irvine, CA (US); Stanley Kyle Hayes, Mission Viejo, CA (US); Joey Camia Reglos, Lake Forest, CA (US); Robert Gutierrez, Huntington Beach, CA (US); J. Christopher Flaherty, Topsfield, MA (US)

Correspondence Address: RIMAS LUKAS;VERTIFLEX, INC., 1954 KELLOGG AVE., SUITE 100, CARLSBAD, CA, 92008, US

(21) Appl. No.: 11/801,319

(22) Filed: May 9, 2007

Related U.S. Application Data
(63) Continuation-in-part of application No. 11/427,738, filed on Jun. 29, 2006.
Application No. 11/427,738 is a continuation-in-part of application No. 11/436,407, filed on May 17, 2006, now abandoned.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
Application No. 11/427,738 is a continuation-in-part of application No. 11/362,366, filed on Feb. 23, 2006.
(63) Continuation-in-part of application No. 11/586,849, filed on Oct. 25, 2006.
Application No. 11/586,849 is a continuation-in-part of application No. 11/362,366, filed on Feb. 23, 2006.
(63) Continuation-in-part of application No. 11/726,093, filed on Mar. 20, 2007.
Application No. 11/726,093 is a continuation-in-part of application No. 11/586,849, filed on Oct. 25, 2006.
Application No. 11/586,849 is a continuation-in-part of application No. 11/362,366, filed on Feb. 23, 2006.
Application No. 11/726,093 is a continuation-in-part of application No. 11/427,736, filed on Jun. 29, 2006.
Application No. 11/427,738 is a continuation-in-part of application No. 11/436,407, filed on May 17, 2006, now abandoned.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
(60) Provisional application No. 60/701,660, filed on Jul. 22, 2005.
(60) Provisional application No. 60/701,660, filed on Jul. 22, 2005.
(60) Provisional application No. 60/701,660, filed on Jul. 22, 2005.

(51) Int. Cl. A61B 17/58

(52) U.S. Cl. 606/64

(57) ABSTRACT

A spinal cross-connector for connecting two stabilization rods installed in a patient's spine is provided. The cross-connector includes novel rod attachment elements dynamically connected together by connector elements. The cross-connector provides multi-dimensional adjustability for easy and accurate installation with full lock-down.

### CROSS-REFERENCE TO RELATED APPLICATIONS

This application is a continuation-in-part of U.S. patent application Ser. No. 11/427,738, filed on Jun. 29, 2006, entitled “Systems and methods for posterior dynamic stabilization of the spine”, which is a continuation-in-part of U.S. patent application Ser. No. 11/436,407, filed on May 17, 2006, entitled “Systems and methods for posterior dynamic stabilization of the spine”, which is a continuation-in-part of U.S. patent application Ser. No. 11/033,452, filed on Jan. 10, 2005, entitled “Systems and methods for posterior dynamic stabilization of the spine”, which is a continuation-in-part of U.S. patent application Ser. No. 11/006,495, filed on Dec. 6, 2004, entitled “Systems and methods for posterior dynamic stabilization of the spine”, which is a continuation-in-part of U.S. patent application Ser. No. 10/970,366, filed on Oct. 20, 2004, entitled “Systems and methods for posterior dynamic stabilization of the spine”. U.S. patent application Ser. No. 11/427,738, filed on Jun. 29, 2006, entitled “Systems and methods for posterior dynamic stabilization of the spine” is a continuation-in-part of U.S. patent application Ser. No. 11/362,366, filed on Feb. 23, 2006, entitled “Systems and methods for stabilization of bone structures”, which claims priority to U.S. Provisional Patent Application Ser. No. 60/701,660, filed on Jul. 22, 2005, entitled “Systems and methods for stabilization of bone structures”. This application is also a continuation-in-part of U.S. patent application Ser. No. 11/726,093, filed on Mar. 20, 2007, entitled “Screw systems and methods for use in stabilization of bone structures”, which is a continuation-in-part of U.S. patent application Ser. No. 11/586,849, filed on Oct. 25, 2006, entitled “Systems and methods for stabilization of bone structures”, which is a continuation-in-part of U.S. patent application Ser. No. 11/362,366, filed on Feb. 23, 2006, entitled “Systems and methods for stabilization of bone structures”, which claims priority to U.S. Provisional Patent Application Ser. No. 60/701,660, filed on Jul. 22, 2005, entitled “Systems and methods for stabilization of bone structures”. U.S. patent application Ser. No. 11/726,093, filed on Mar. 20, 2007, entitled “Screw systems and methods for use in stabilization of bone structures” is also a continuation-in-part of U.S. patent application Ser. No. 11/427,738, filed on Jun. 29, 2006, entitled “Systems and methods for posterior dynamic stabilization of the spine”, which is a continuation-in-part of U.S. patent application Ser. No. 11/436,407, filed on May 17, 2006, entitled “Systems and methods for posterior dynamic stabilization of the spine”, which is a continuation-in-part of U.S. patent application Ser. No. 11/033,452, filed on Jan. 10, 2005, entitled “Systems and methods for posterior dynamic stabilization of the spine”, which is a continuation-in-part of U.S. patent application Ser. No. 11/006,495, filed on Dec. 6, 2004, entitled “Systems and methods for posterior dynamic stabilization of the spine”, which is a continuation-in-part of U.S. patent application Ser. No. 10/970,366, filed on Oct. 20, 2004, entitled “Systems and methods for posterior dynamic stabilization of the spine”. This application is also a continuation-in-part of U.S. patent application Ser. No. 11/726,093, filed on Mar. 20, 2007, entitled “Screw systems and methods for use in stabilization of bone structures”, which is a continuation-in-part of U.S. patent application Ser. No. 11/586,849, filed on Oct. 25, 2006, entitled “Systems and methods for stabilization of bone structures”, which is a continuation-in-part of U.S. patent application Ser. No. 11/362,366, filed on Feb. 23, 2006, entitled “Systems and methods for stabilization of bone structures”, which claims priority to U.S. Provisional Patent Application Ser. No. 60/701,660, filed on Jul. 22, 2005, entitled “Systems and methods for stabilization of bone structures”. All of the above applications are claimed for their benefit of priority and are further incorporated herein by reference in their entirety.

### FIELD

The present invention is directed towards the treatment of spinal disorders and pain. More particularly, the present invention is directed to systems and methods of treating the spine which reduce pain and enable spinal motion, and which effectively mimic that of a normally functioning spine.

### BACKGROUND

FIGS. 1A and 1B illustrate a portion of the human spine having a superior vertebra 2 and an inferior vertebra 4, with an intervertebral disc 6 located in between the two vertebral bodies. The superior vertebra 2 has superior facet joints 8a and 8b, inferior facet joints 10a and 10b, posterior arch 16 and spinous process 18. Pedicles 3a and 3b interconnect the respective superior facet joints 8a, 8b to the vertebral body 2. Extending laterally from superior facet joints 8a, 8b are transverse processes 7a and 7b, respectively. Extending between each inferior facet joint 10a and 10b and the spinous process 18 are lamina 5a and 5b, respectively. Similarly, inferior vertebra 4 has superior facet joints 12a and 12b, superior pedicles 9a and 9b, transverse processes 11a and 11b, inferior facet joints 14a and 14b, lamina 15a and 15b, posterior arch 20, spinous process 22.

The superior vertebra with its inferior facets, the inferior vertebra with its superior facets, the intervertebral disc, and seven spinal ligaments (not shown) extending between the superior and inferior vertebrae together comprise a spinal motion segment or functional spine unit. Each spinal motion segment enables motion along three orthogonal axes, both in rotation and in translation. The various spinal motions are illustrated in FIGS. 1C-1E. In particular, FIG. 1C illustrates flexion and extension motions and axial loading, FIG. 1D illustrates lateral bending motion and translation, and FIG. 1E illustrates axial rotational motion. A normally functioning spinal motion segment provides physiological limits and stiffness in each rotational and translational direction to create a stable and strong column structure to support physiological loads.

Traumatic, inflammatory, metabolic, synovial, neoplastic and degenerative disorders of the spine can produce debilitating pain that can affect a spinal motion segment's ability to properly function. The specific location or source of spinal pain is most often an affected intervertebral disc or facet joint, and in particular the nerves in and around the intervertebral disc or facet joint. Often, a disorder in one location or spinal component can lead to eventual deterioration or disorder, and ultimately, pain in another.

Spine fusion (arthrodesis) is a procedure in which two or more adjacent vertebral bodies are fused together once the natural height of the degenerated disc has been restored. It is one of the most common approaches to alleviating various types of spinal pain, particularly pain associated with one or more affected intervertebral discs. However, fusion is only as good as the ability to restore disc height to relieve the pain by taking pressure off the nerves, nerve roots, and/or articulating surfaces—i.e., facet joints and end plates of the vertebral bodies.

One way of accomplishing fusion is to install pedicles screws in adjacent vertebral bodies, followed by installation of fusion rods between the screws. This type of system can be strengthened by attaching a cross-connector between the fusion rods. In many current systems, however, attachment and deployment of such a cross-connector is difficult.

With the limitations of current spine stabilization technologies, there is clearly a need for an improved means and methods for stabilization of the spine which addresses the drawbacks of prior devices. In particular, it would be highly beneficial to have a fusion stabilization system that has high strength and that enables the spine to mimic the motion of one or more healthier, uncompromised vertebral segments, especially with regard to torsional motions. It would be additionally beneficial if such a system could be conveniently installed and used to treat various spinal indications regardless of pain source, prevent or slow the deterioration of the intervertebral discs, or even restore disc height, and be used in conjunction with prosthetic intervertebral discs.

### SUMMARY OF THE INVENTION

According to one aspect of the invention, a spinal stabilization system is provided. The system includes a first rod attachment element configured to connect to a first vertebral stabilization rod. A second rod attachment element configured to connect to a second vertebral stabilization rod. The system includes a first bar attached to the first rod attachment element and a second bar attached to the second rod attachment element. The system includes a connector connecting the first and second bars. At least one rod attachment element has a two-part design such that one part of the two-part design contacts one portion of a corresponding rod and the other part of the two-part design contacts another portion of the corresponding rod to capture the rod.

According to another aspect of the invention, a spinal stabilization system for a patient is provided. The system includes a first rod attachment element configured to connect to a first vertebral stabilization rod. The first rod attachment element includes a first biasing section. The system includes a second rod attachment element configured to connect to a second vertebral stabilization rod. The second rod attachment element includes a second biasing section. The system includes a first bar connected to the first rod attachment element and a second bar connected to the second rod attachment element. A connector is provided that connects the first and second bars. At least the first rod attachment element further comprises a rod-contacting surface and a corresponding screw having a head with a cam section such that rotation of the screw having a head with a cam section into the rod attachment element forces the cam section towards the rod-contacting surface capturing the rod between the rod-contacting surface and the cam section.

According to another aspect of the invention, a spinal stabilization system for a patient is provided. The system includes a first rod attachment element configured to connect to a first vertebral stabilization rod and a second rod attachment element configured to attach to a second vertebral stabilization rod. A cross-connector configured to connect the first and second rod attachment elements is also provided. At least one rod attachment element has a two-part design such that one part of the two-part design contacts one portion of a corresponding rod and the other part of the two-part design contacts an other portion of the corresponding rod to capture the rod.

According to another aspect of the invention, a method for stabilizing a patient's spine is provided in which a first set of two pedicle screw systems is installed into a superior vertebral segment. A second set of two pedicle screw systems is installed into an inferior vertebral segment. A first rod is connected between one of the pedicle screw systems in the first set and one of the pedicle screw systems in the second set. A second rod is connected between the other of the pedicle screw systems in the first set and the other of the pedicle screw systems in the second set. A first rod attachment element is connected to the first rod and a second rod attachment element is connected to the second rod. A first bar is connected to the first rod attachment element and a second bar is connected to the second rod attachment element. A cross connector is connected to both the first bar and the second bar. At least one rod attachment element has a two-part design such that one part of the two-part design contacts one portion of a corresponding rod and the other part of the two-part design contacts an other portion of the corresponding rod to capture the rod.

According to another aspect of the invention, a method for stabilizing a patient's spine is provided in which a first set of two pedicle screw systems is installed into a superior vertebral segment. A second set of two pedicle screw systems is installed into an inferior vertebral segment. A first rod is connected between one of the pedicle screw systems in the first set and one of the pedicle screw systems in the second set. A second rod is connected between the other of the pedicle screw systems in the first set and the other of the pedicle screw systems in the second set. A first rod attachment element is connected to the first rod and a second rod attachment element is connected to the second rod. A cross-connector is connected between the first and second rod attachment elements. At least one rod attachment element has a two-part design such that one part of the two-part design contacts one portion of a corresponding rod and the other part of the two-part design contacts another portion of the corresponding rod.

According to another aspect of the invention, a spinal stabilization system for a patient is provided. The system includes a first rod attachment element configured to attach to a first vertebral stabilization rod and a second rod attachment element configured to attach to a second vertebral stabilization rod. The system includes a cross-connector configured to connect the first and second rod attachment elements. At least one rod attachment element has a two-part design such that one part of the two-part design contacts one portion of a corresponding rod and the other part of the two-part design contacts an other portion of the corresponding rod to capture the rod. The one and the other parts of the two-part design move relative to each other upon the tightening of a screw.

According to another aspect of the invention, a spinal stabilization system for a patient is provided. The system includes a first rod attachment element configured to attach to a first vertebral stabilization rod and a second rod attachment element configured to attach to a second vertebral stabilization rod. A cross-connector configured to connect the first and second rod attachment elements is provided. At least one rod attachment element has a two-part design such that a first part of the two-part design contacts a circumferential portion of a corresponding rod along a portion thereof, and the other part of the two-part design contacts a portion of the first part. The one and the other parts of the two-part design move relative to each other upon the tightening of a screw, and movement of the other part causes the first part to tighten around the circumferential portion of the rod.

According to another aspect of the invention, a spinal stabilization system for a patient is provided. The system includes a first rod attachment element configured to attach to a first vertebral stabilization rod and a second rod attachment element configured to attach to a second vertebral stabilization rod. A cross-connector coupled between the first and second rod attachment elements is provided. The first and second rod attachment elements define openings for capturing first and second vertebral stabilization rods, and the openings face in a substantially anterior direction when the rods are being captured.

According to another aspect of the invention, a method for stabilizing a patient's spine is provided. The method includes the step of installing a first set of two pedicle screw systems into a superior vertebral segment. A second set of two pedicle screw systems is installed into an inferior vertebral segment. A first rod is connected between one of the pedicle screw systems in the first set and one of the pedicle screw systems in the second set. A second rod is connected between the other of the pedicle screw systems in the first set and the other of the pedicle screw systems in the second set. A first rod attachment element is connected to the first rod, and a second rod attachment element is connected to the second rod. At least one of the first and second rod attachment elements is connected by moving the rod attachment element, having an anteriorly-facing opening, towards the rod, such that the rod enters the opening in the rod attachment element in a posterior direction.

According to another aspect of the invention, a method for stabilizing a patient's spine is provided. In the method, a first set of two pedicle screw systems is installed into a superior vertebral segment. A second set of two pedicle screw systems is installed into an inferior vertebral segment. A first rod is connected between one of the pedicle screw systems in the first set and one of the pedicle screw systems in the second set. A second rod is connected between the other of the pedicle screw systems in the first set and the other of the pedicle screw systems in the second set. A first rod attachment element is connected to the first rod, and a second rod attachment element is connected to the second rod. A cross-connector is connected between the first and second rod attachment elements. A screw is provided in the cross-connector. The method includes the step of and the cross-connector is configured such that tightening one screw in the cross-connector such that the one tightening prevents all polyaxial and/or translational movement of the cross-connector relative to the first and second rod attachment elements.

According to another aspect of the invention, a spinal stabilization system for a patient is provided. The system includes a first rod attachment element configured to attach to a first vertebral stabilization rod and a second rod attachment element configured to attach to a second vertebral stabilization rod. A cross-connector attached between the first and second rod attachment elements is provided. The system is configured such that the cross-connector is displaced by a predetermined distance in a posterior direction relative to a point where at least one rod attachment element attaches to a bar. The displacement accommodates the shape of the anatomy and bridges anatomy located anterior of the cross-connector.

According to another aspect of the invention, a spinal stabilization system for a patient is provided. The system includes a first rod attachment element configured to attach to a first vertebral stabilization rod and a second rod attachment element configured to attach to a second vertebral stabilization rod. A cross-connector attached between the first and second rod attachment elements is provided. The first rod attachment element defines an interior surface that is configured to encompass a rod along as great a percentage of a circumference of the rod as possible while allowing the rod to be snap-fit into the rod attachment element.

According to another aspect of the invention, a method for stabilizing a patient's spine is provided. In the method, a first set of two pedicle screw systems is installed into a superior vertebral segment and a second set of two pedicle screw systems is installed into an inferior vertebral segment. A first rod is connected between one of the pedicle screw systems in the first set and one of the pedicle screw systems in the second set. A second rod is connected between the other of the pedicle screw systems in the first set and the other of the pedicle screw systems in the second set. A first rod locking procedure is performed by connecting a first rod attachment element to the first rod in a snap-fit manner. A second rod locking procedure is performed by connecting a second rod attachment element to the second rod in a snap-fit manner.

According to another aspect of the invention, a centered spinal stabilization system for a patient is provided. The system includes a first rod attachment element configured to attach to a first vertebral stabilization rod and a second rod attachment element configured to attach to a second vertebral stabilization rod. A cross-connector attached between the first and second rod attachment elements is provided. The first rod attachment element defines a channel having a rod-contacting surface that is configured to engage a rod and to center the rod in the channel when the rod is fully engaged.

According to another aspect of the invention, a spinal stabilization system for a patient is provided. The system includes a first rod attachment element to attach to a first vertebral stabilization rod and a second rod attachment element to attach to a second vertebral stabilization rod. A cross-connector coupled between the first and second rod attachment elements is provided. The first and second rod attachment elements define a first and second channel having first and second rod-contacting surfaces for capturing first and second vertebral stabilization rods. At least one of the first rod-contacting surface and second rod-contacting surface includes a gripping surface along a portion thereof.

According to another aspect of the invention, a spinal stabilization system for a patient is provided. The system includes a first rod attachment element configured to attach to a first vertebral stabilization rod and a second rod attachment element configured to attach to a second vertebral stabilization rod. A cross-connector coupled between the first and second rod attachment elements is provided. The cross-connector is coupled to the first rod attachment element by a first screw and to the second rod attachment element by a second screw. At least one of these couplings includes a slot such that the corresponding screw can slide a distance along the slot prior to tightening of the screw to allow for variations in patient anatomy.

According to another aspect of the invention, a method for stabilizing a patient's spine is provided. In the method, a first set of two pedicle screw systems is installed into a superior vertebral segment and a second set of two pedicle screw systems is installed into an inferior vertebral segment. A first rod is connected between one of the pedicle screw systems in the first set and one of the pedicle screw systems in the second set and a second rod is connected between the other of the pedicle screw systems in the first set and the other of the pedicle screw systems in the second set. A first rod attachment element is connected to the first rod and a second rod attachment element is connected to the second rod. A cross-connector is connected between the first and second rod attachment elements such that the cross-connector is coupled to the first rod attachment element by a first screw and to the second rod attachment element by a second screw, and at least one of the couplings includes a slot wherein the corresponding screw can slide a distance along the slot prior to tightening to allow for variations in patient anatomy.

According to another aspect of the invention a spinal stabilization system for a patient is provided. The system includes a first rod attachment element configured to attach to a first vertebral stabilization rod and a second rod attachment element configured to attach to a second vertebral stabilization rod. A cross-connector is configured to connect the first and second rod attachment elements and has a locked configuration and an unlocked configuration such that the cross-connector moves relative to at least one of the first and second rod attachment elements while in the unlocked configuration.

According to another aspect of the invention, a spinal stabilization system for a patient is provided. The system includes a first rod attachment element configured to attach to a first vertebral stabilization rod and a second rod attachment element configured to attach to a second vertebral stabilization rod. A first bar is provided and connected to the first rod attachment element. The first bar extends towards the second rod attachment element. A second bar is provided and connected to the second rod attachment element and extends towards the first rod attachment element. One of the first or second bars overlaps at least a portion of the other of the first or second bars and a cross-connector is provided to connect the first and second bars together at the overlapping portion.

Advantages of the invention may include one or more of the following. Devices according to embodiments of the invention may be easily installed once other spinal components are installed, such as screws, rods, dynamic elements, facet constructs, and so on. The cross-connector system allows ease of operator assembly and surgical placement, and allows multi-degree-of-freedom adjustability prior to final stabilization. The cross-connector system further allows repositioning in subsequently-performed procedures. Devices according to embodiments of the invention may have a low profile and be minimally invasive.

Systems according to the invention may be employed to treat various spinal disorders and pain, including those involving degenerative disc disease, spinal stenosis, spondylolisthesis, spinal deformities, fractures, pseudarthrosis, tumors, failed fusions, arthritic facet joints, severe facet joint tropism, facet joint injuries, deformed facet joints, scoliosis, and other vertebral segment traumas and diseases.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1(A)-(B) illustrate certain aspects of the anatomy of spinal segments.

FIGS. 1(C)-(E) illustrate various spinal movements that may be performed by the spinal segments of FIGS. 1(A)-(B).

FIG. 2(A) illustrates a side schematic view of a cross-connector with accompanying rod attachment elements and rods according to a first embodiment of the invention.

FIG. 2(B) illustrates an exploded perspective view of a cross-connector according to the first embodiment of the invention.

FIG. 3 illustrates a side view of a cross-connector according to the first embodiment of the invention.

FIG. 4 illustrates a side schematic view, in partial cross-section, of a cross-connector system according to a second embodiment of the invention.

FIGS. 5(A)-(D) illustrate exploded and non-exploded perspective and side cross-sectional views of a cross-connector according to a third embodiment of the invention.

FIG. 6 illustrates a perspective view of a cross-connector according to a fourth embodiment of the invention.

FIGS. 7(A)-(B) illustrate perspective and exploded views of a cross-connector according to a fifth embodiment of the invention.

FIG. 8 illustrates a perspective exploded view of a cross-connector according to a sixth embodiment of the invention.

FIGS. 9(A)-(E) illustrate various views of a cross-connector system according to a seventh embodiment of the invention.

FIGS. 10(A)-(C) illustrate sectional perspective, top, and side views of the cross-connector system according to the seventh embodiment of the invention.

FIGS. 11(A)-(B) illustrate side sectional and perspective views of the cross-connector system according to the seventh embodiment of the invention.

FIG. 12 illustrates an exploded perspective view of a cross-connector system according to an eighth embodiment of the invention.

FIGS. 13(A)-(C) illustrate sectional perspective, top, and side views of the cross-connector system according to the eighth embodiment of the invention.

FIGS. 14(A)-(C) illustrate top, bottom, and detailed views of the cross-connector system according to the eighth embodiment of the invention.

FIGS. 15(A)-(C) illustrate perspective and side views, in partial cross-section, of a cross-connector system according to a ninth embodiment of the invention.

FIGS. 16(A)-(C) illustrate more detailed views of the cross-connector system according to the ninth embodiment of the invention.

FIGS. 17(A)-(C) illustrate more detailed views of the cross-connector system according to the ninth embodiment of the invention.

FIGS. 18(A)-(D) illustrate more detailed views of the cross-connector system according to the ninth embodiment of the invention.

FIGS. 19(A)-(B) illustrate side and perspective exploded views of a cross-connector system according to a tenth embodiment of the invention.

FIGS. 20(A)-(C) illustrate more detailed views of the cross-connector system according to the tenth embodiment of the invention.

FIGS. 21(A)-(B) illustrate more detailed views of the cross-connector system according to the tenth embodiment of the invention.

FIG. 22 illustrates a detailed view of an alternative cross-connector system related to the tenth embodiment of the invention.

FIGS. 23(A)-(B) illustrate side and perspective exploded views of a cross-connector system according to an eleventh embodiment of the invention.

FIG. 24 illustrates a more detailed view of the cross-connector system according to the eleventh embodiment of the invention.

FIGS. 25(A)-(B) illustrate side and perspective exploded views of a cross-connector system according to a twelfth embodiment of the invention.

FIGS. 26(A)-(B) illustrate more detailed views of the cross-connector system according to the twelfth embodiment of the invention.

FIGS. 27(A)-(B) illustrate side and perspective exploded views of a cross-connector system according to a thirteenth embodiment of the invention.

FIGS. 28(A)-(C) illustrate more detailed views of the cross-connector system according to the thirteenth embodiment of the invention.

FIGS. 29(A)-(B) illustrate more detailed views of the cross-connector system according to the thirteenth embodiment of the invention.

FIG. 30 illustrates a detailed view of an alternative cross-connector system related to the thirteenth embodiment of the invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

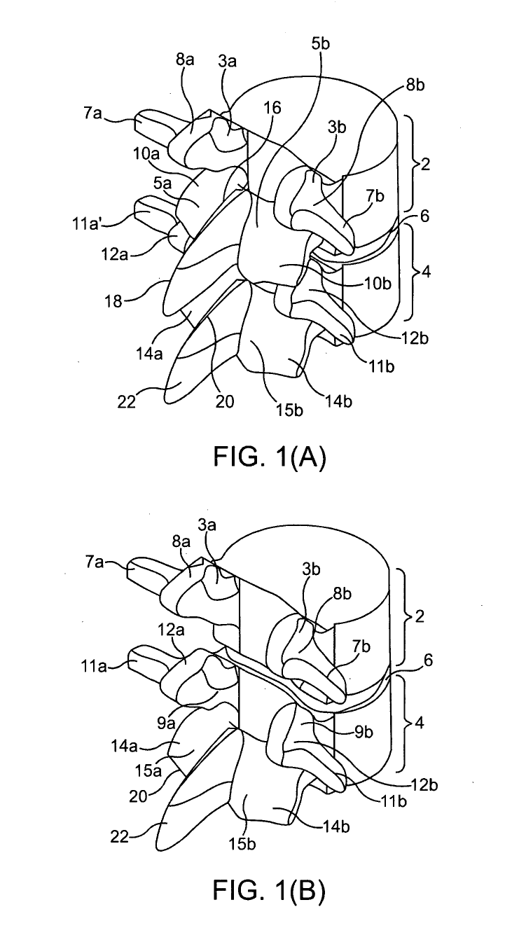

The present invention will now be described in greater detail by way of the following description of exemplary embodiments and variations of the systems and methods of the present invention. While more fully described in the context of the description of the subject methods of implanting the subject systems, it should be initially noted that in certain applications where the natural facet joints are compromised, as illustrated in FIG. 1(A), inferior facets 10a and 10b, lamina 5a and 5b, posterior arch 16 and spinous process 18 of superior vertebra 2 may be resected for purposes of implantation of certain of the dynamic stabilization systems of the present invention. In other applications, where possible, the natural facet joints, lamina and/or spinous processes are spared and left intact for implantation of other dynamic stabilization systems of the present invention.

It should also be understood that the term “system”, when referring to a system of the present invention, most typically refers to a set of components which includes a superior, cephalad or rostral (towards the head) component configured for implantation into a superior vertebra of a vertebral motion segment and an inferior or caudal (towards the feet) component configured for implantation into an inferior vertebra of a vertebral motion segment. A pair of such component sets includes one set of components configured for implantation into and stabilization of the left side of a vertebral segment and another set configured for the implantation into and stabilization of the right side of a vertebral segment. The left set of components may move independently of the right set of components or their motions may be coordinated via an attachment between the two. In other words, they may move in conjunction with one another, with both moving relative to the more fixed attachment between the two. Many of the systems disclosed here concern such an attachment between the two.

Where multiple spinal segments or units are being treated, the term “system” may refer to two or more pairs of component sets, i.e., two or more left sets and/or two or more right sets of components. Such a multilevel system involves stacking of component sets in which each set includes a superior component, an inferior component, and one or more medial components therebetween. These multilevel systems may include cross member or cross connector components or strut systems having differing properties, e.g., lengths, limits on travel or other limited ranges of motion; resistance to motion or other forces, attachment locations, etc.

The superior and inferior components (and any medial components therebetween), when operatively implanted, are engaged or interface with each other in a manner that enables the treated spinal motion segment to mimic the function and movement of a natural healthy segment. The disclosed systems include one or more structures or members which enable, limit and/or otherwise selectively control spinal motion. The structures may perform such functions by exerting various forces on the system components, and thus on the target vertebrae. The manner of coupling, interfacing, engagement or interconnection between the subject system components may involve compression, distraction, rotation or torsion, or a combination thereof. In certain embodiments, the extent or degree of these forces or motions between the components may be intraoperatively selected and/or adjusted to address the condition being treated, to accommodate the particular spinal anatomy into which the system is implanted, and to achieve the desired therapeutic result, such as to restore disc height and offset the facet joints.

In certain embodiments, the superior and inferior components are mechanically coupled to each other by one or more interconnection or interfacing means. In other embodiments, the superior and inferior components interface in an engaging manner which does not necessarily mechanically couple or fix the components together but rather constrains their relative movement and also enables the treated spinal motion segment to mimic the natural function and movement of a healthy segment. Typically, the interconnecting means is a posteriorly-positioned component, i.e., one positioned posteriorly of the superior and inferior components, or it may be a laterally-positioned component, i.e., one positioned to the outer side of the posterior and inferior components. The structures may involve one or more strut systems and/or joints which provide for dynamic movement of a stabilized spinal motion segment.

In this description, the following terms are used throughout, and are defined here. A “cross-connector system” is a device that extends between and attaches to two fixation or stabilization rods. A “rod attachment element” forms a portion of a cross-connector system, and is the portion of the cross-connector system that attaches to the rod. The portion of the cross-connector system, that is not the rod attachment element, is the cross connector itself.

It is noted that the following patent applications, owned by the assignee of the present invention and incorporated herein by reference in their entirety for all purposes, disclose various dynamic rod systems, pedicle screw systems, and facet augmentation systems that may be employed in conjunction with the current invention: U.S. patent application Ser. No. 11/427,738, filed on Jun. 29, 2006, entitled “Systems and methods for posterior dynamic stabilization of the spine”; U.S. patent application Ser. No. 11/436,407, filed on May 17, 2006, entitled “Systems and methods for posterior dynamic stabilization of the spine”; U.S. patent application Ser. No. 11/033,452, filed on Jan. 10, 2005, entitled “Systems and methods for posterior dynamic stabilization of the spine”; U.S. patent application Ser. No. 11/006,495, filed on Dec. 6, 2004, entitled “Systems and methods for posterior dynamic stabilization of the spine”; U.S. patent application Ser. No. 10/970,366, filed on Oct. 20, 2004, entitled “Systems and methods for posterior dynamic stabilization of the spine”; U.S. patent application Ser. No. 11/726,093, filed on Mar. 20, 2007, entitled “Screw systems and methods for use in stabilization of bone structures”; U.S. patent application Ser. No. 11/586,849, filed on Oct. 25, 2006, entitled “Systems and methods for stabilization of bone structures”; U.S. patent application Ser. No. 11/362,366, filed on Feb. 23, 2006, entitled “Systems and methods for stabilization of bone structures”; U.S. Provisional Patent Application Ser. No. 60/701,660, filed on Jul. 22, 2005, entitled “Systems and methods for stabilization of bone structures”; all of which are incorporated by reference herein in their entirety.

In many of the systems described, the adjustability of the system may be used to prevent undesired stress on spine and system components. The adjustability also provides for a simplified installation process. To this end, the requirements for precision on drilling locations, angles, etc., may be reduced. In the same way, an installation kit may be provided with a lesser number of components, as the provided components can accommodate more varying anatomy. Thus, in many systems, the components are aligned to the anatomy and then tightened.

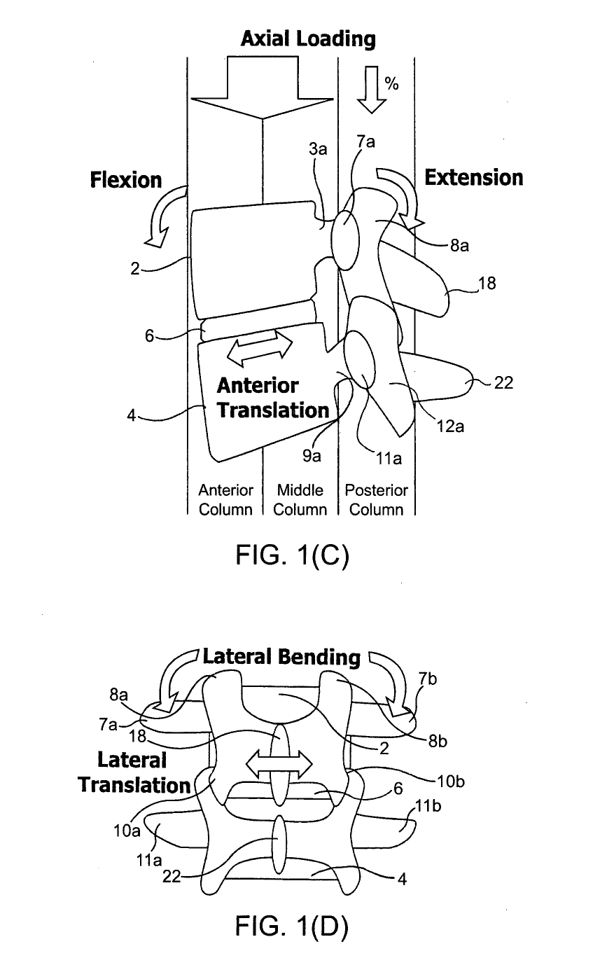

FIG. 2(A) shows a high-level design of a top-loading cross-connector system 51 in side view. The cross-connector system 51 includes a cross connector 50 and two rod attachment elements 52a and 52b. Bars 68a and 68b extend from the rod attachment elements 52a and 52b toward a connector 56. Two rods 70a and 70b are shown as well, with the cross-connector system 51 in a loading position above the rods. As may be seen in the figure, rod attachment elements 52a and 52b each have arch-shaped channels 53a and 53b defined therein to engage rods 70a and 70b, respectively.

Bars 68a and 68b may be individually or collectively formed integral with or pre-attached to either rod attachment elements 52a and 52b, or with connector 56, or both. This alternative is true for any of the embodiments described in this application, unless otherwise noted.

In use, and in general for all of the systems, pedicle screw systems are installed in the pedicles of a patient, and rods 70a and 70b, as well as additional rods or less rods as necessary, are installed between the pedicle screws. Once the rods are installed, one or more rod attachment elements are affixed to the rods. If the rod attachment elements include bars and/or connectors in a pre-attached or integral fashion, then the technique is completed following attachment of the rod attachment elements or bars to any unattached elements. If the rod attachment elements do not include bars and/or connectors in a pre-attached or integral fashion, then the bars are attached to the rod attachment elements. If at least one bar includes a connector in a pre-attached or integral fashion, then the technique is completed following any necessary affixation of the connector to either bar. If the bars do not include a connector in a pre-attached or integral fashion, then the connector is attached to the bars, and the technique is completed. For this system and for the other cross-connector systems described, once an implantation procedure is completed, all components may be fixed relative to one another, preventing relative motion. Alternatively, one or more components may be secured and yet relative motion may still be allowed, such as resiliently-biased motion via a dynamic element or otherwise. Motions may also be permitted such as simple rotation or sliding motions, such as via a spring-biased attachment. One example of this may be the sliding swivels described in embodiments below. When the cross-connector components are tightened, all swivel motion may be prevented or alternatively some limited motions may continue to be allowed, in one or more directions.

FIG. 2(B) shows a cross-connector system 51 in more detail. The cross-connector system 51 includes rod attachment elements 52a and 52b, as well as a cross connector 50. A two-part rod attachment element 52a includes (the rod attachment element 52b has similar corresponding elements, though each rod attachment element may be different if dictated by the application) a clamp assembly having a biasing section or hook 76a and a pivoting clamp 78a.

In this and in other embodiments, the “two-part rod attachment element” refers to a rod attachment element in which one element or a portion of one element is moved toward another element in order to secure a rod therebetween, but generally neither of these elements, in this embodiment, is a screw, though a screw may be used to move one element towards another element. Moving one element towards another may include either translation or rotational motion or both. Moving one element towards another may tend to provide a clamping action or the like. It should be noted that “moving one element towards another” refers to any action that can secure a rod, such as the above described clamping action. Indeed, the actual amount that elements are moved towards each other may be minimal (and parts of the elements may even move away from each other), and substantial movement is not required. In another embodiment, described below, one of the elements is a screw with a cam portion.

A locking screw 82a serves to, upon installation, force the pivoting clamp 78a towards hook 76a, thereby clamping the hook against the pivoting clamp and thus securing the rod attachment element to the rod 70a. It should be noted that the hook need not clamp against the pivoting clamp in an extreme sense; it may be sufficient that the hook pivots to reduce the internal diameter of the arch-shaped channels such that the hook and pivoting clamp capture and apply force to the rod.

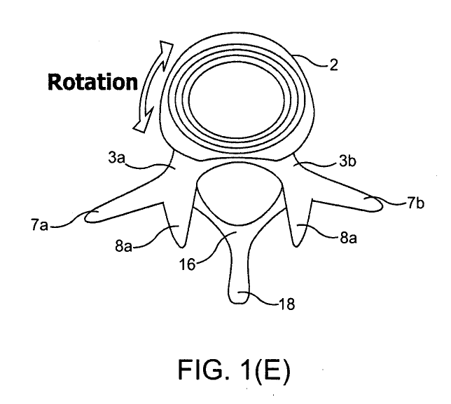

In more detail, the hook 76a includes two side pieces 57a and 59a joined at a central piece 61a. Hingedly attached to the hook 76a is the pivoting clamp 78a. A hinge 86a is shown in FIG. 3 to demonstrate this attachment. Also shown in FIG. 3 is the hole 63a defined in pivoting clamp 78a into which the screw 82a is installed. As may be seen in that figure, a bottom surface 69a of the head of the screw 82a forms an angle 65a with the top surface 67a of the hook 76a. Downward translation of the screw 82a forces surfaces 69a and 67a together (reducing angle 65a), and causes rotation of the hook 76a in the direction indicated by arrow 80, the rotation being about the hinge 86a. When the hook 76a rotates in this direction, a rod-contacting portion 88a of the rod attachment element clamps down on the rod 70a, securing it against movement. The rod-contacting portion 88a of the rod attachment element preferably encircles the rod 70a by greater than 180°.

As noted, bar 68a extends from a central section 55a of the rod attachment element 52a, and may be integral with or pre-attached to the same. The central section is installed within the biasing or hook section 76a, but may also be integral therewith.

In an alternative embodiment, the bar 68a may be installed in (e.g. screwed into) rod attachment element 52a after rod attachment element 52a is attached to the rod 70a. Bar 68a includes a swivel 72a that translates along a groove 84a. The swivel 72a, at its radial extreme, approximates a spherical shape. When disposed in a corresponding approximately-spherical cavity within opening 74a in cross connector 56, the swivel's shape allows a degree of polyaxial movement or adjustment of each rod attachment element relative to the cross connector. For example, the rod attachment element may rotate about an axis 60 parallel to the longitudinal axis of the cross connector. This may be particularly important when accommodation is necessary for non-parallel rods. Of course, following such polyaxial adjustment, the system may be tightened down, prohibiting future movements, for most fusion procedures. As another adjustment mechanism, the groove 84a allows a constrained degree of translational movement and adjustment along axis 60. That is, the bar may move in or out of the swivel, to accommodate various spacings between rods.

The cross connector 56 includes a connector top 62 and a connector bottom 58. The connector top 62 and the connector bottom 58 engagedly mate and are affixed via an bar clamping screw 64 which is installed through a hole 66a defined in connector top 62 and which is threaded into a threaded hole 66b defined in connector bottom 58. The bar clamping screw or the threaded hole may be provided with an anti-rotation feature, such as a nylon insert or metal swage.

Once the bar clamping screw is installed in holes 66a and 66b, further movement of the swivels along the groove, as well as polyaxial motion, may be prohibited. Alternatively, installation of the bar clamping screw 64 may only serve to prevent removal, while allowing one or both of these motions.

While the above embodiment has been described with respect to the rod attachment element with elements having “a” suffixes, a similar description applies to the rod attachment element with elements having “b” suffixes.

In use, after the rods 70a and 70b are attached to the installed pedicle screws, the rod attachment elements above may be attached to the rods by placing the rods against rod-contacting portion 53a (a corresponding portion, 53b, is not shown), and tightening screws 82a and 82b into holes 63a and 63b (hole 63a is indicated in FIG. 3). The bars 68a and 68b along with swivels 72a and 72b may then be disposed in the voids 74a and 74b of the cross connector 56, i.e., in the connector bottom 58, with the swivels located along the grooves 84a and 84b. The connector top 62 is then placed above the connector bottom 58 and the screw 64 is inserted through the hole 66a and is threaded into hole 66b. Once tightened, the system is secured and the procedure concluded. In an alternative embodiment, the connector top and connector bottom are secured first, or partially secured first, and then the rod attachment elements are secured to the rods.

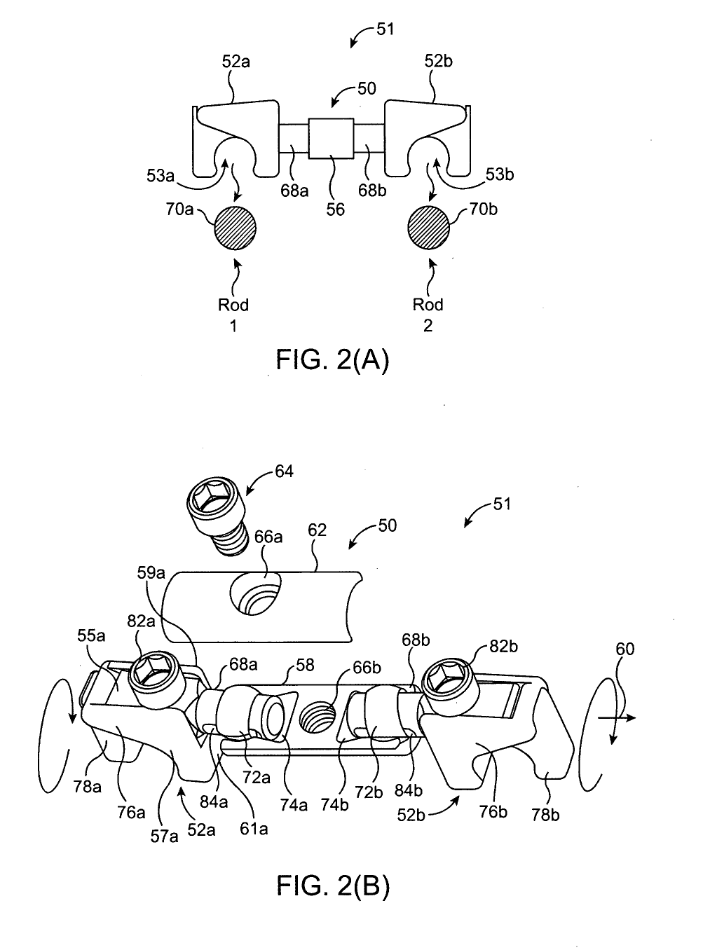

FIG. 4 shows a second embodiment of the invention, with some elements in common with the embodiment of FIGS. 2-3. A cross-connector system 90 is shown with two-part rod attachment elements 92a and 92b, and a cross connector 91. In this embodiment, the cross connector 91 includes a dynamic element 114. The dynamic element 114 may include any type of element that can provide a degree of motion to the cross connector 91, including the types of dynamic elements disclosed in U.S. patent Ser. No. 11/427,738. For example, the dynamic element may provide a resilient bias, such as with a flexible portion or a spring. One or more characteristics of dynamic element 91 may be adjustable (adjustment means not shown but may be, e.g., a rotatable set screw), such as an adjustment to the range of motion and/or a force applied to resist motion.

The cross connector 91 further includes depending cylindrical projections 112a and 112b, these depending from opposite sides of the dynamic element 114. Into each cylindrical projection 112a and 112b may be placed corresponding bars 106a and 106b, respectively. As in the first embodiment, the bars 106a and 106b have disposed thereon swivels 108a and 108b. The swivels 108a and 108b may slide along, and/or pivot within, a groove as in the first embodiment (not shown in FIG. 4).

FIG. 4 shows the bars 106a and 106b as threadingly engaging the rod attachment elements 92a and 92b, though may also be constructed integral to the same. If threadingly engaged, they may be pre-attached before the surgical installation procedure or attachment may be contemporaneous, during the surgical installation procedure.

In FIG. 4, the rod attachment element 92a is displayed as being of a different construction from the rod attachment element 92b. In more detail, the rod attachment element 92a includes dual screws 94a and 96a that may be employed to grasp a rod at rod-contacting surface 116a. While not indicated in FIG. 4, they may act in a way similarly to that of FIG. 3, in which the bottom surface of the screw head contacting the top surface of the rod attachment element causes a pivoting action, closing the rod attachment element around the rod. In an alternative embodiment (not shown), the two screws 94a and 96a may each contact an interior surface of the clamp assembly. This contact may then deflect the contacted surface in a way to clamp around the rod. The screw 94a deflects the left side and screw 96a deflects the right side. In any case, the two screws 94a and 96a may cause rod-contacting portion 116a to close in a complimentary fashion around the rod.

The rod attachment element 92a is also shown with a drug delivery element 104. The drug delivery element 104 may be appropriately configured to provide a time-release of, e.g., an antibiotic drug, and may be refillable via an injection port integral to drug delivery element 104 (injection port not shown). Such a drug delivery element may be provided or performed on any of the described embodiments.

The rod attachment element 92b also has some similarities to the rod attachment element 52b, with the following differences. First, either the screw 94b or the rod attachment element 92b may be provided with a nylon insert 98 to provide an anti-rotation function. The nylon insert 98 may be replaced with a metal swage or the like to perform a similar function. The rod attachment element 92b also incorporates a cover 102 to cover the head of the screw 94b. Such a contamination cover may be provided on any of the described embodiments. The cover 102 may be replaced with a degree of filling of the hole, such as by an elastomer. Either will serve to help prevent tissue in-growth, or the ingress of other forms of contamination. Keeping this area free of contamination may provide significant assistance in post-procedural adjustment or removal. Another difference between the rod attachment elements 92b and 52b is that 92b uses a single screw 94b to activate clamping function.

In use, after the rods 70a and 70b are attached to the installed pedicle screws, the rod attachment elements above may be attached to the rods by placing the rods against rod-contacting portions 116a and 116b, and tightening screws 94a, 94b, and 96a into their respective holes. The bars 106a and 106b along with swivels 108a and 108b may then be disposed in the voids of the cross connector 91 in any of the manners disclosed above or below. The bars may be pre-installed in the cross connector during, e.g., the time of construction of the dynamic element. Once tightened, the system is secured and the procedure concluded. In an alternative embodiment, the bars are secured to the cross connector first, and then the rod attachment elements are secured to the rods. In another alternative embodiment, dynamic element 114 is adjusted such as at a time prior to, during and/or after implantation of cross-connector system 90.

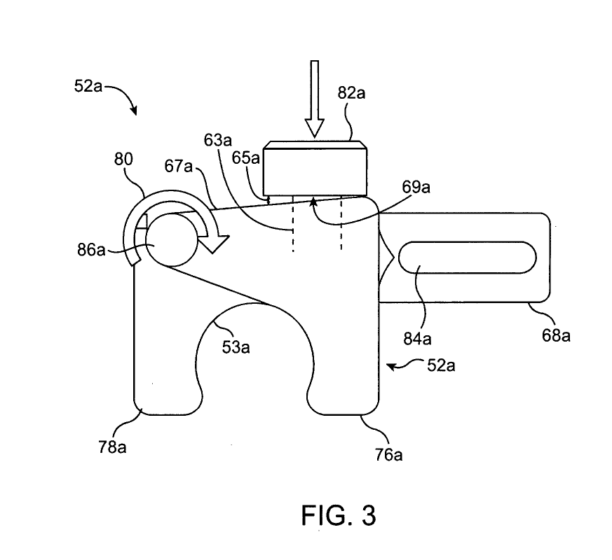

FIG. 5(A)-(D) illustrates a cross-connector system 120 according to a third embodiment of the invention, this embodiment incorporating certain features of the aforedescribed embodiments.

In FIG. 5(A), two stabilizing rods 110a and 110b are engaged by two corresponding two-part rod attachment elements 118a and 118b. The rod attachment elements 118a and 118b each have a biasing section or hook section 126a and 126b, respectively, which operate in conjunction with sliding clamps 128a and 128b to grasp rods 110a and 110b.

In more detail, sliding clamps 128a and 128b each have corresponding hook-engaging elements 132a and 132b (see FIG. 5(D)) which are slidingly received by corresponding holes defined in the hook sections 126a and 126b. At the opposite end of each of sliding clamps 128a and 128b is a section defining an upwardly-facing recess 134a and 134b. Two rod-locking screws 138a and 138b are provided to tighten the sliding clamps 128a and 128b to the hook sections 126a and 126b, and this tightening is accomplished by the rod-locking screws 138a and 138b each being installed in holes 142a and 142b and then respectively engaging the recesses 134a and 134b (note 134b is not shown in the figure). That is, once the hook-engaging elements 132a and 132b are slidingly received by the corresponding holes defined in the hook sections 126a and 126b, they cannot be forced downward any further, and the downward pressure of the rod-locking screws 138a and 138b then serves to frictionally engage and make secure the connection between the hook sections and the clamps, as well as closing around the rods. The presence of the recesses tends to secure the clamps in a predetermined position relative to the hook sections, this predetermined position chosen to ensure sufficient force is applied against the rods 110a and 110b to secure the same against movement.

The cross connector 121 as shown in FIG. 5(C) includes a body section 123 from which depends two clamp sections 122a and two clamp sections 122b. The two clamp sections 122a and two clamp sections 122b each form a “C” clamp, and each has a hole defined therein through which screws 124a and 124b may be inserted to tighten the respective clamp sections. A swivel 144b is shown in FIG. 5(C) in a position in which the same may be inserted into the cross connector 121. A bore of the swivel allows entry into the swivel of a bar. Following insertion, the swivel 144b may be rotated such that it can no longer be removed from the cross connector 121 under normal motions encountered by the cross-connector system 120 in normal patient use.

In use, after the rods 110a and 110b are attached to the installed pedicle screws, the rod attachment elements may be attached to the rods by placing the rods between the hook and the clamp sections, and tightening screws 138a and 138b into holes 142a and 142b. The bars 136a and 136b along with swivels 144a and 144b may then be disposed in the voids of the cross connector 121. That is, the bars are inserted into the swivels, rotated, and then advanced into the voids of cross connector 121>. The screws 124a and 124b may then be installed and tightened, securing the bars and swivels against further movement. Once tightened, the system is secured and the procedure concluded. In an alternative embodiment, the cross connector 121 is constructed and secured first, and then the rod attachment elements are secured to the rods.

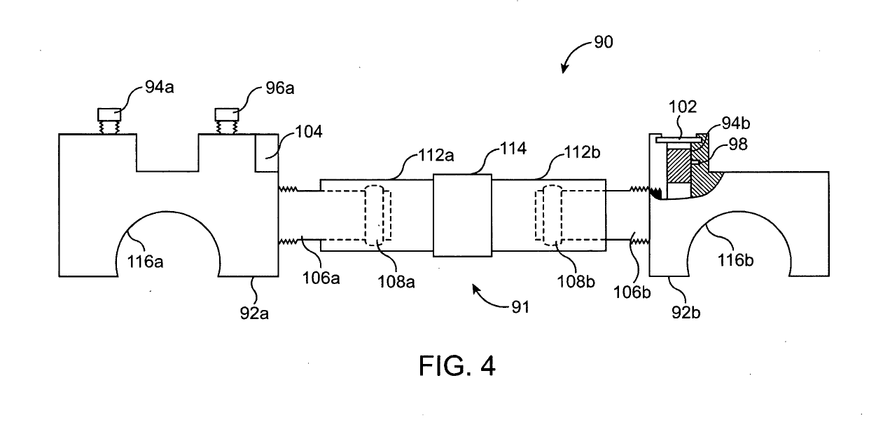

FIG. 6 shows an embodiment of the invention, similar to that of FIG. 5(A)-(D), in which a single screw provides the compressive force. In particular, two rods 110a and 110b are attached to two two-part rod attachment elements 131a and 131b, respectively, via two respective screws 138a and 138b. Of course, other attachment mechanisms can also be employed. The rod attachment elements 131a and 131b each have a corresponding bar 127a and 127b, which form a part of a cross-connector system 141. The cross-connector system 141 also includes a cross connector portion 146, formed of a wrap-around partial cylindrical portion 147 which is attached in its general mid-section to a top projecting portion 152a and a bottom projecting portion 152b, which when forced together by a screw 148 tends to frictionally hold the bars 127a and 127b in a predetermined and desired relationship.

In use, the system of FIG. 6 is constructed in a manner similar to that of FIG. 5(A)-(D), except that only one bar clamping screw need be tightened.

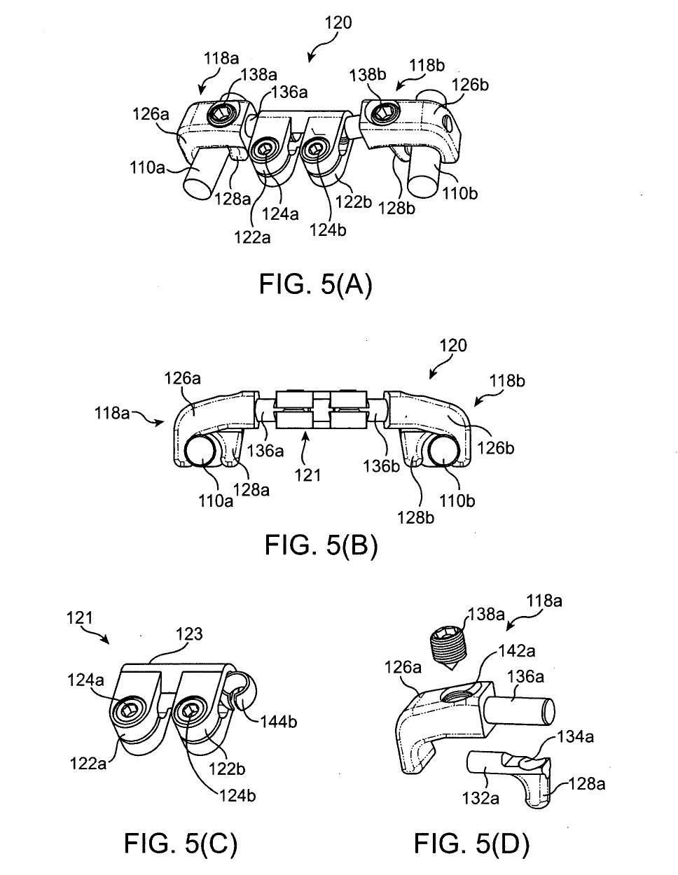

FIGS. 7(A) and 7(B) show a related embodiment, in which a two-piece cross connector 139 includes an upper housing 159 with a projecting mid-portion. While the reference numerals for common elements remain the same as in FIG. 6, changed elements include the upper housing 159 which engages a lower housing 154. The upper housing 159 and lower housing 154 are held together via a screw 162. The screw 162 is inserted through a downwardly-projecting section 158 that defines a hole therethrough. The upper housing 159 has a first portion 156a which engages a bar 127a and a second portion 156b which engages a bar 127b. In both cases, the first and second portions primarily engage their respective corresponding bars via contacting the swivels that are slid onto the bars, though the first and second portion may in some cases also contact the bars themselves. The screw 162 is inserted through a hole 158 in the upper housing 159 and is threaded into a threaded hole 161 in the lower housing 154.

In use, after the rods 110a and 110b are attached to the installed pedicle screws, the rod attachment elements may be attached to the rods by placing the rods between the hook and the clamp sections, and tightening screws 138a and 138b into their respective holes. The bars 127a and 127b along with their corresponding swivels may then be disposed in the voids of the cross connector 139, i.e., in the lower housing 154. The upper housing 159 is then placed above the lower housing 154 and the screw 162 is inserted through the hole 158 and is threaded into hole 161. Once tightened, the system is secured and the procedure concluded. In an alternative embodiment, the upper and lower housings are secured first, and then the rod attachment elements are secured to the rods.

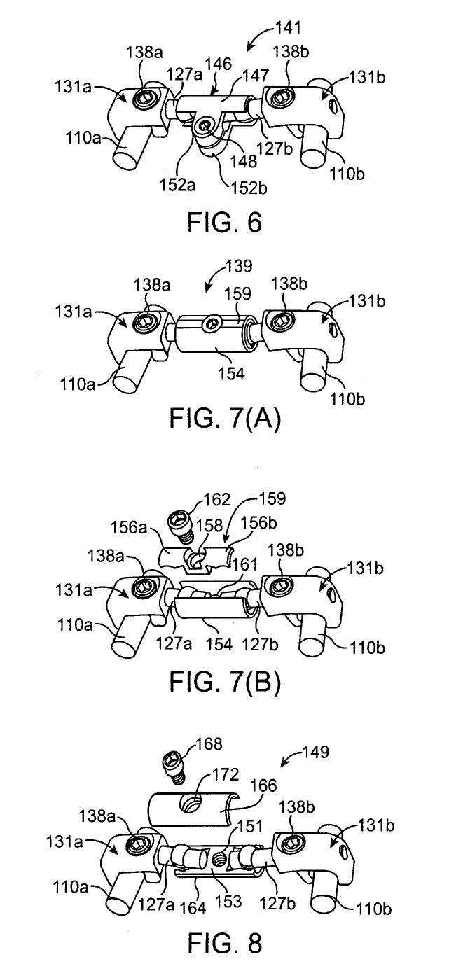

FIG. 8 shows a related embodiment. Whereas the embodiment of FIGS. 7(A) and (B) included an upper housing with a downwardly-projecting mid-portion where the downwardly-projecting mid-portion stabilizes internal components, the embodiment of FIG. 8 includes a lower housing with a upwardly-projecting mid-portion, this upwardly-projecting mid-portion similarly capable of stabilizing internal components. In particular, the cross-connector system 149 includes an upper housing 166 and a lower housing 164, the lower housing having a raised mid-portion 153 in which is defined a threaded hole 151. A screw 168 is inserted through a hole 172 in the upper housing 166 and is threaded into the threaded hole 151.

In use, the system of FIG. 8 is constructed in a manner similar to that of FIG. 7(A)-(B).

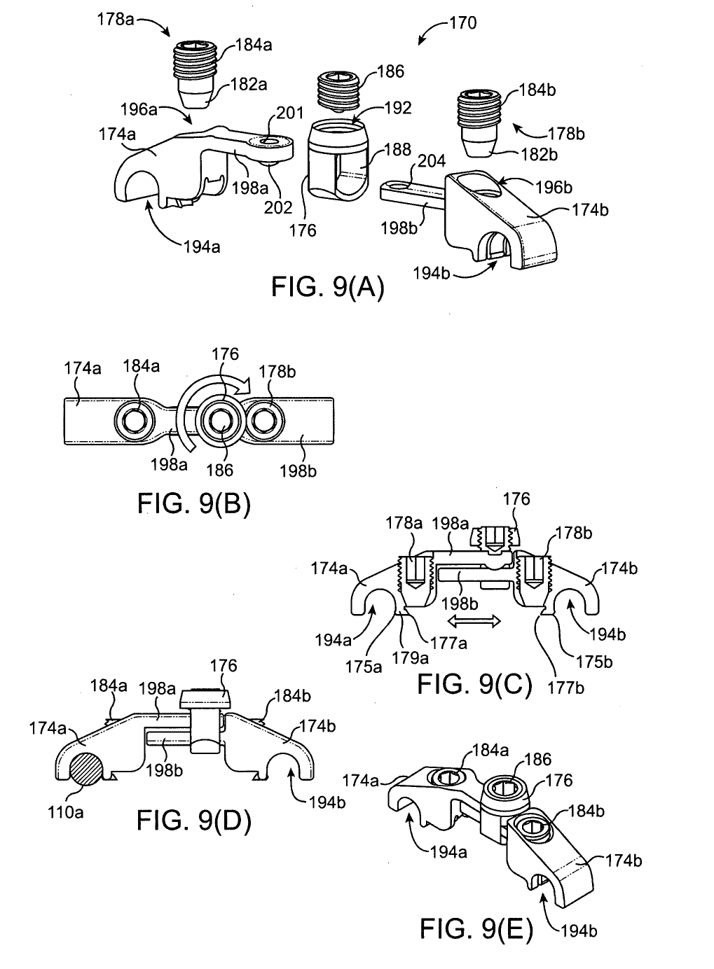

FIGS. 9(A)-(E) illustrate another embodiment of the invention. In this embodiment, as will be described, the rod attachment element snaps over a rod and a screw insertion closes a clamp around the rod. In addition, the bars include upper and lower bars with portions that overlap, slide and mate with each other. In this embodiment, the cross connector may be formed of a single piece that surrounds both the upper and lower bars at a single cross-sectional location. A screw may directly contact the bars, compressing them together.

In more detail, and referring initially to FIG. 9(A), a cross-connector system 170 includes first and second rod attachment elements 174a and 174b, each having a respective rod-contacting portion 194a and 194b for contacting rods (a rod 110a is shown in FIG. 9(D)). The rod-contacting portions may be generally sized such that the same contact the rods around as great a percentage of the rods as possible. Of course, if the rod-contacting portions are sized to extend around too great a circumference, the rods would not be able to be installed within the rod-contacting portions in a snap-fit fashion; in this case, pre-installation or pre-engagement would be necessary. Moreover, the rod-contacting portions are generally circular in cross-section. The radius of the circle described may be chosen such that the rod is automatically centered when the rod is installed in the rod attachment element. That is, in most embodiments the rod should not move around within the rod-contacting portion of the rod attachment element. In many cases, this means that the center of the rod-contacting portion and the center of the rod are substantially coincident when the rod is installed. The above features of the rod-contacting portions may be extended to various other embodiments in this description.

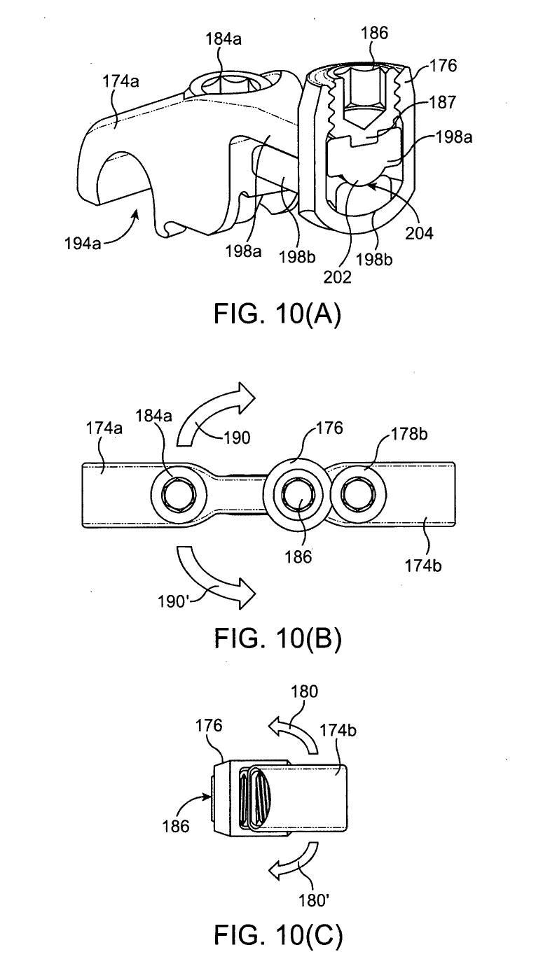

The rod attachment elements 174a and 174b have corresponding bars 198a and 198b. The bars 198a and 198b are configured to attach to their respective rod attachment elements at different heights, so that one may be slid on top of another when the two are each inserted through a hole 188 in a unitary cross connector 176. The hole 188 may have an appropriate shape to allow a substantial clamping effect when an bar clamping screw 186 is threadingly inserted through a hole 192 defined in the top of the cross connector 176. A distal end 187 of the screw 186 may mate with a corresponding recess in the top of the bar 198a (see FIG. 10(A)). The tolerances of the bars in the hole 188 may be such as to allow a degree of rotation, as best seen in FIG. 9(E). In other words, the bars need not be exactly collinear. The allowed rotation may be about an axis defined by a nub 202 in the bar 198a which mates with a recess 204 in the bar 198b (see FIG. 10(A)).

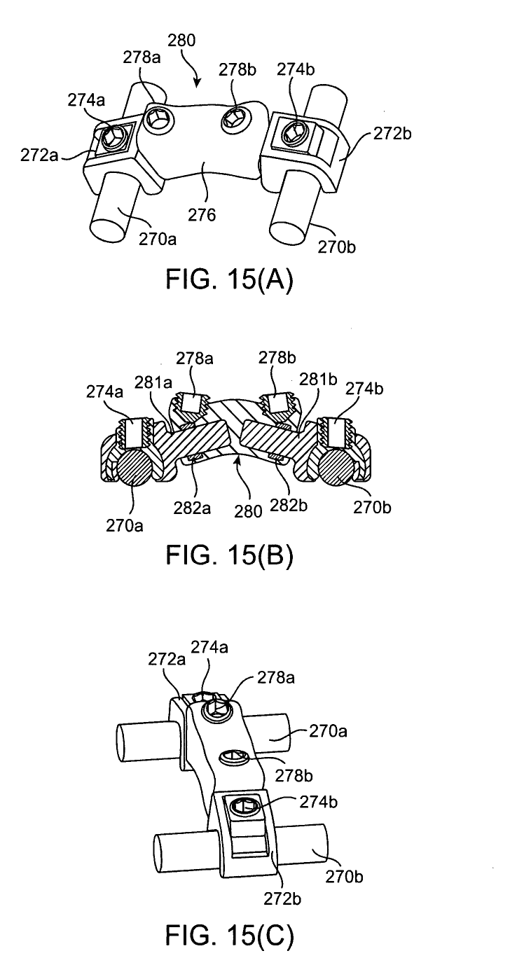

As may be seen in FIG. 9(D), the cross-connector is displaced a certain distance in a posterior direction from a position where the rod attachment element attaches to a rod. This displacement allows the system to accommodate the shape of and bridge the anatomy in the vertebral region. That is, the bars 198a and 198b are above the line defined by the cross-sectional midpoints of rods 110a and 110b at the point of attachment to the cross-connector 170. FIGS. 15(A)-(C) show a related embodiment, where the bridge-like accommodation is provided by the cross-connecting forming a domed shape.

Referring back to FIGS. 9(A)-(D), the rod-contacting surfaces 194a and 194b may surround the rod by greater than 180°, and may be provided with a roughened surface or coating so as to enhance the same's grip on the rod. The roughened surface may be accomplished via grit-blasting the surface, defining knurling, serrations, or splines thereon, or the like, and the same may be provided or performed on any of the described embodiments. Besides increasing the grip, various other advantages may inure to embodiments including serrations or the like. For example, the removal of material from the rod-contacting surface may allow the rod to slide more easily due to decreased friction. Similarly, the removal may allow the rod attachment element, or its biasing or hook section, to flex more easily.

Referring to FIG. 9(C), at an inner extremity 179 of the rod-contacting surface 194a, two opposite-facing projections may be provided. Of course, the same may be provided on the rod attachment element 174b. A first projection 175 may project in a direction such that the first projection 175 further circumferentially surrounds a rod 110a disposed adjacent the rod-contacting surface 194a. The presence of this first projection 175 may also be such that the rod 110a, when placed adjacent the rod-contacting surface 194a, in fact “snap-fits” into the volume defined by the same. This snap-fit may in some cases be sufficient attachment of the rod attachment element to the rod. In many cases, however, this snap-fit will not be sufficient but will serve to help the clinician to precisely position and adjust the connector allowing movement of the connector relative to the rod before completely locking down the device to the rod.

One way of increasing the grip of the rod attachment element on the rod is via use of a second projection 177. Two screws 178a and 178b are provided, each with respective threads 184a and 184b and respective tapering portions 182a and 182b, for insertion into the rod attachment elements 174a and 174b in holes 196a and 196b As best seen in FIG. 9(C), as the screw 178a is inserted into the hole 196a, the leading edge of the screw, adjacent the tapering portion 182a, contacts the second projection 177 and deflects the same in a direction away from the screw 178a, i.e., towards the rod 110a. In particular, the second projection 177 is deflected under the rod 110a, surrounding the rod a greater angular distance and increasing the level of contact and pressure between the rod attachment element and the rod, further frictionally securing the rod against the rod attachment element.

As noted above, a certain degree of rotation is allowed in the system to accommodate situations where the bars are required to be non-collinear. The amount of allowed rotation can vary and can be predetermined based on various factors, especially the width of the bars, their width at their distal tips, and the width of the cross connector 176. This type of alignment, which may be intraoperative, is indicated by arrows 190 and 190′ in FIG. 10(B). The tolerances of the bars and the hole 188 may further allow for a degree of rotation out of the plane defined by arrows 190 and 190′, i.e., in directions defined by arrows 180 and 180′ in FIG. 10(C). Generally, various movements, such as rotation, translation, etc., are usually prevented by further tightening of the associated screws prior to completion of the procedure.

The cross connector 176 may include screw threads 192 which have an anti-rotation feature, or the screw 186 may have an anti-rotation feature, as has been described in connection with other screws above. The tip of the bar clamping screw 186 may engagingly mate with a recess 201 on the upper surface of the bar 198a, i.e., the surface opposite that of nub 202.

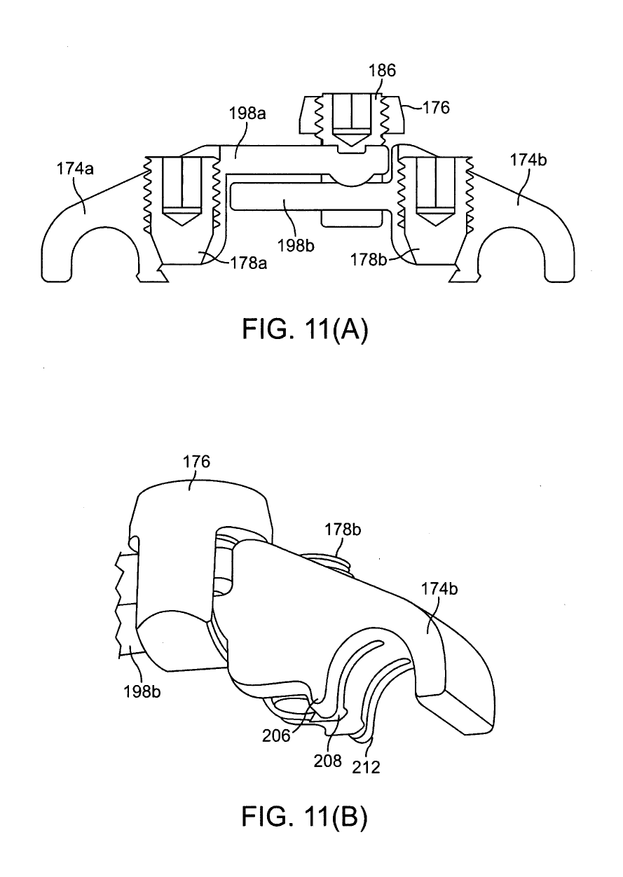

FIG. 11(A) indicates the embodiment in cross-section, as well as how the cross connector may be slid along the bars to accommodate various placement locations. To further assist the engagement of the bars as the cross connector is translated, the bars may have a number of nubs and recesses to accommodate various placement locations (just one nub and recess is shown in FIG. 11(A) for clarity).

FIG. 11(B) indicates an alternative embodiment of the rod attachment element 174b. In FIG. 11(B), the rod attachment element 174b is composed in part of a slotted arrangement that makes up part of the rod-contacting surface. The central section 208 performs the functions described above in connection with FIGS. 9 and 10. In addition, as the central section 208 is separated from the remainder of the rod attachment element, at least in the region of the rod-contacting surface, the second projection may be easier and more convenient to deflect. The remainder of the rod attachment element, in the region of the rod-contacting surface, comprises a set of peripheral sections 206 and 212 which provide additional strength to the rod attachment element.

Referring back to FIG. 9(A)-(E), after the rods 110a and 110b are attached to the installed pedicle screws, the rod attachment elements may be attached to the rods by placing the rods against rod-contacting surfaces 194a and 194b, and tightening screws 178a and 178b into their respective holes. The tightening of screws 178a and 178b flexes undercuts 175a and 175b further under the rod, further securing the same against movement. The bars 198a and 198b are then inserted in an overlapping fashion into the void 188 of cross connector 176, such that the nub 202 engages the recess 204. A degree of orientation may be performed by the physician, to accomplish a particular treatment goal, following which the screw 186 is threadingly inserted into the hole 192. Once tightened, the system is secured and the procedure concluded. In an alternative embodiment, the bars are engaged to the cross connector first, and then the rod attachment elements are secured to the rods.

In many insertion procedures, the screws are inserted and tightened to a point where the same are not fully tightened. Following this, the system can be adjusted according to the preferences of the physician, and then the screws fully tightened to prevent undesired motion.

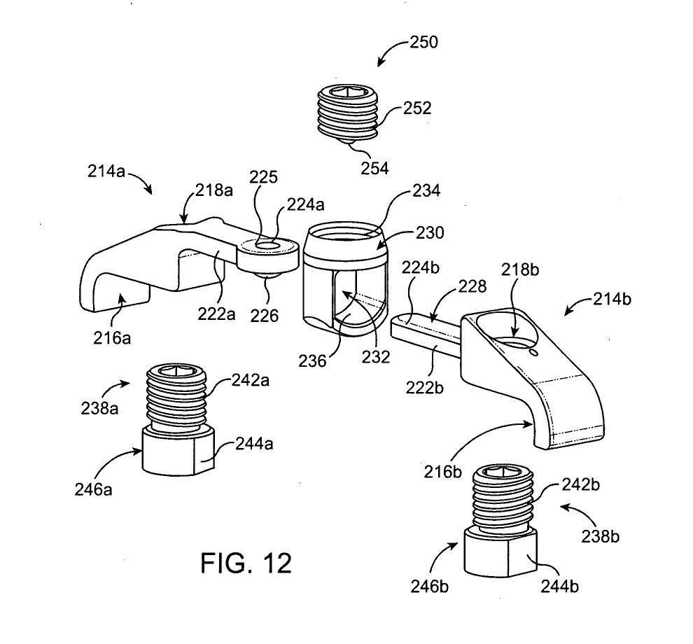

Referring to FIG. 12, an alternative embodiment of a cross-connector system is shown. Certain features are in common with above-described embodiments. For example, two rod attachment elements 214a and 214b are shown, each with a corresponding bar 222a and 222b. The bar 222a has a nub 226 on a lower portion of a distal end 224a while, on an upper portion, the same has a recess 225. The bar 222b has a recess 228 along a portion of its length. When the two bars are inserted into a hole 232 in cross connector 230, the same may be tightened into position by inserting a bar clamping screw 250 having tip 254 and threads 252 into threaded hole 234.

Each rod attachment element has a threaded hole 218a (or 218b) and a biasing section or hook section 216a (or 216b). The hook section has a concave surface for contacting a portion of a rod (not shown). Two rod attachment element screws 238a and 238b are provided, one each for threading engagement with corresponding holes 218a and 218b. The two rod attachment element screws 238a and 238b have respective threads 242a and 242b and respective heads 244a and 244b. The heads 244a and 244b each have a corresponding eccentric cam section 246a and 246b.

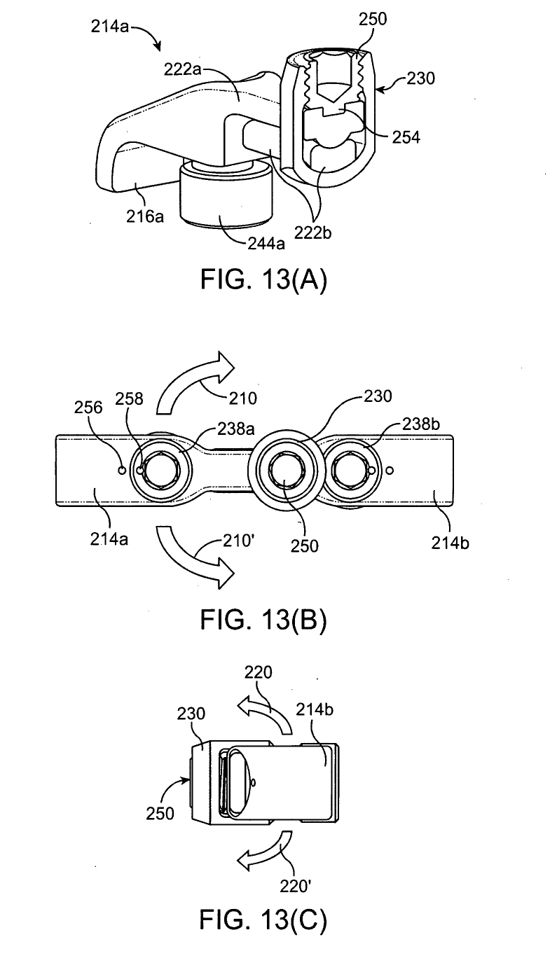

FIG. 13(A) shows a perspective cross-section of this embodiment's configuration. Referring to FIG. 13(B), a degree of rotational movement or adjustment may be allowed as indicated by arrows 210 and 210′. In addition, a degree of rotational movement or adjustment may be allowed, out of the plane defined by arrows 210 and 210′, this degree of rotational movement indicated by FIG. 13(C) as arrows 220 and 220′. As noted above, once the proper adjustment is made, generally for reasons of patient geometry accommodation, the system is tightened, preventing further movement. Of course, various degrees of freedom may be non-tightened if desired to allow movement with respect to that degree of freedom.

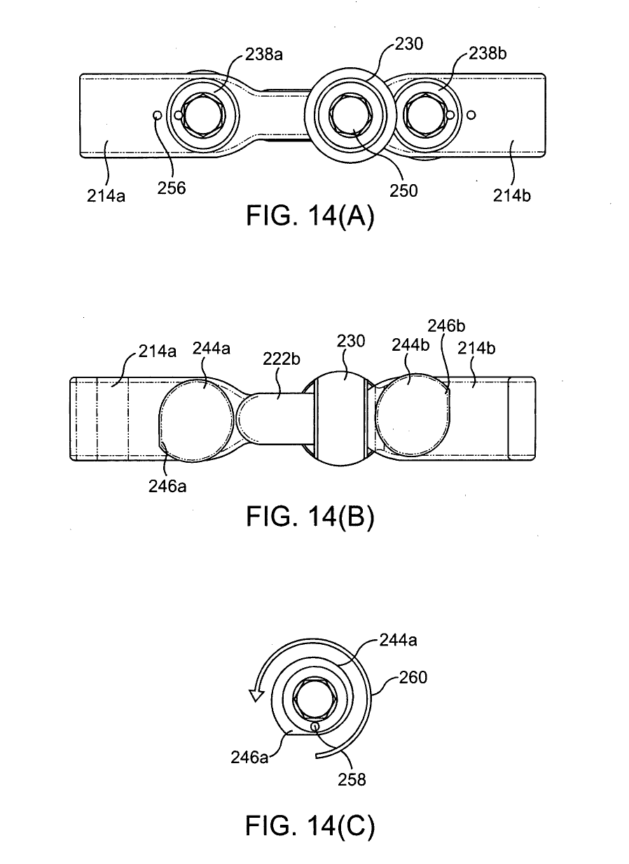

The cam position may be indicated by markers 256 and 258, located on the bar and on the screws (see FIG. 13(B)). Top and bottom views are also shown in FIGS. 14(A) and (B). A detail of the head 244a is shown in FIG. 14(C). This figure shows cam section 246a, marker 258, as well as intended direction of rotation 260, for left-handed threads. The marker on the screw and the marker on the bar may be employed to align starting positions, ending positions, etc. For example, aligned markers may indicate a starting position, where the cam is not engaged with the rod, and a 90° rotation may then be employed to capture the rod.

A captured rodis placed in juxtaposition with the cross-connector in a functional manner, such as for example, in juxtaposition with the rod attachment element of the cross-connector. A captured rod may be permitted free movement, limited movement, or no movement. In some embodiments, and depending on the level to which tightening of, e.g., screws, is performed, the rod may be permitted no movement, sliding movement, limited rotational movement, significant rotational movement, and so on.

In use, after the rods are attached to the installed pedicle screws, the rod attachment elements may be attached to the rods by placing the rods against rod-contacting surfaces 216a and 216b, and tightening screws 238a and 238b into their respective holes. With this tightening, the cam section 246a locks against the rod, both frictionally arresting and mechanically preventing movement of the rod out of the rod attachment element. That is, the cam section 246a may force the rod against the hook section 216, and thus frictionally secure the same against movement. The cam section 246a can also, with appropriate design, take a position under the rod and force the same upward against the rod attachment element, thus mechanically preventing removal, at least removal via a downward motion. The degree of frictional arrest and mechanical movement prevention may be adjusted by choice of geometry of the cam, the rod, and rod attachment element hook section, and to a lesser degree by the type of materials chosen for construction. In all installation techniques, the physician may be aware of the positioning of the cam section via the markers.

The bars 222a and 222b are then inserted in an overlapping fashion into the void 232 of cross connector 236, such that the nub 226 engages the recess 228. A degree of orientation may be performed by the physician, to accomplish a particular treatment goal, following which the screw 250 is threadingly inserted into the hole 234. As noted above, in typical installations, the screw 250 is inserted first, but not fully tightened. The physicians orients the system properly, and then fully tightens screw 250. Once tightened, the system is secured and the procedure concluded. In an alternative embodiment, the bars are engaged to the cross connector first, and then the rod attachment elements are secured to the rods.

FIGS. 15(A)-(C) show another embodiment of the invention. In this embodiment, rods 270a and 270b are shown coupled to two-part rod attachment elements 272a and 272b, this coupling occurring as will be described in a different manner than the above-described embodiments. Rod-locking screws 274a and 274b assist in creating this coupling. A cross connector 280 is shown with a housing 276 and further employing screws 278a and 278b, these screws clamping directly on respective swivels 282a and 282b and/or on respective bars 281a and 281b.

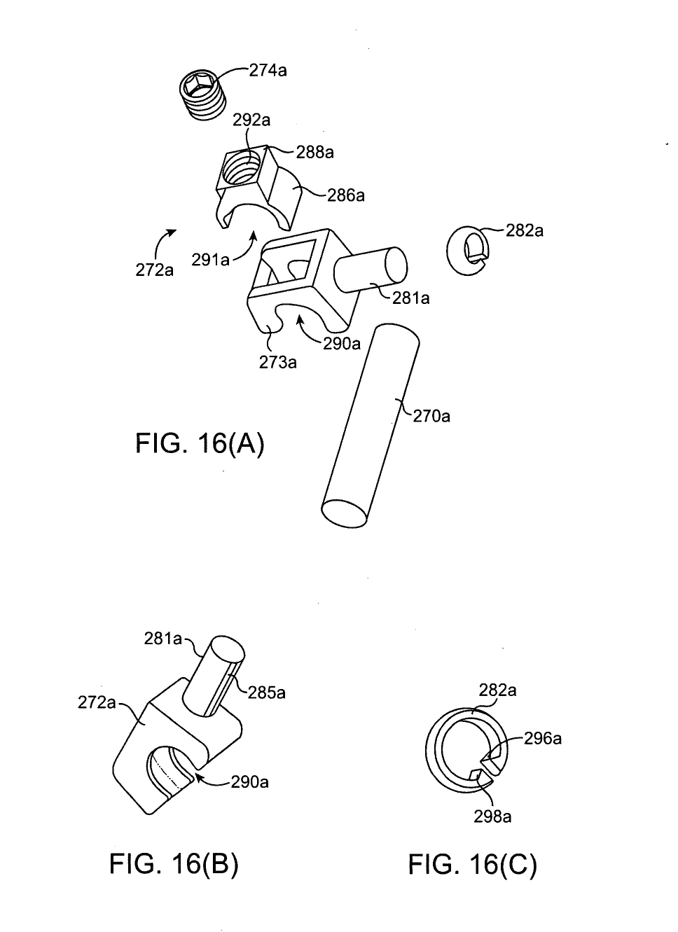

Additional details of this embodiment are shown in FIGS. 16-18.

First, details of the rod-locking screw system are shown in FIG. 16(A)-(B). Referring to FIG. 16(A), the rod attachment element 272a includes a rod-locking screw 274a which is inserted into a clamp 286a. The clamp 286a includes an upwardly-projecting screw receiver 288a with internal threads 292a. The clamp 286a is inserted into a housing 273a, the housing having a rod-receiving channel 290a and a bar 281a, on which is mounted the swivel 282a as will be described. The clamp 286a has a curved rod-receiving lower surface 291a which acts to surround the rod 270a.

Referring to FIG. 16(C), the swivel 282a is mounted on the bar 281a. In particular, radially-inward projections 296a and 298a may together be inserted into a groove 285a in the bar 281a. This engagement may serve as a retaining feature, maintaining the swivel on the bar but still allowing sliding of the swivel on the bar for, e.g., width adjustment.

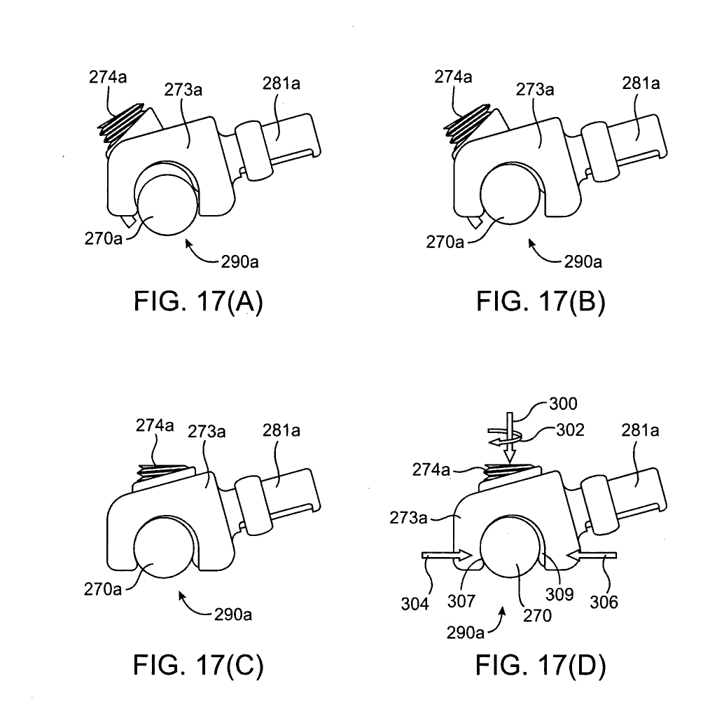

Referring to FIGS. 16(A) and 17(A)-(D), the orientation of the clamp 286a may be adjusted to ease rod insertion. FIG. 17(A) shows the orientation prior to insertion of the rod. The surface 291a and the rod-receiving channel 290a are rotationally-oriented such that the rod may be easily inserted, the configuration just after insertion shown in FIG. 17(B). The clamp 286a may then be rotated as shown in FIG. 17(C), at which point a portion of the clamp is forced against the rod and the same transmits a force against the rod-receiving channel 290a, frictionally securing the components together. That is, in one orientation, first and second portions of the rod attachment element are arranged such that a rod may enter the rod-receiving channel. In another orientation, the first and second portions of the rod attachment element are arranged such that the rod is locked in the rod-receiving channel.

To maintain the frictional engagement, the screw 274a may be rotated in a direction shown by arrow 302, causing a downward movement of the screw indicated by arrow 300. The tightening of the rod-locking screw causes the clamp to compress around the rod, in the directions indicated by arrows 304 and 306, and the tightening may be maintained until the rod is rigidly attached, both axially and rotationally. The rotation of the screw and clamp causes the rod to be clamped between portions 307 and 309 (see FIG. 17(D)). This is termed a “scissor” design. When the screw 274 is tightened, the clamping force is enhanced.

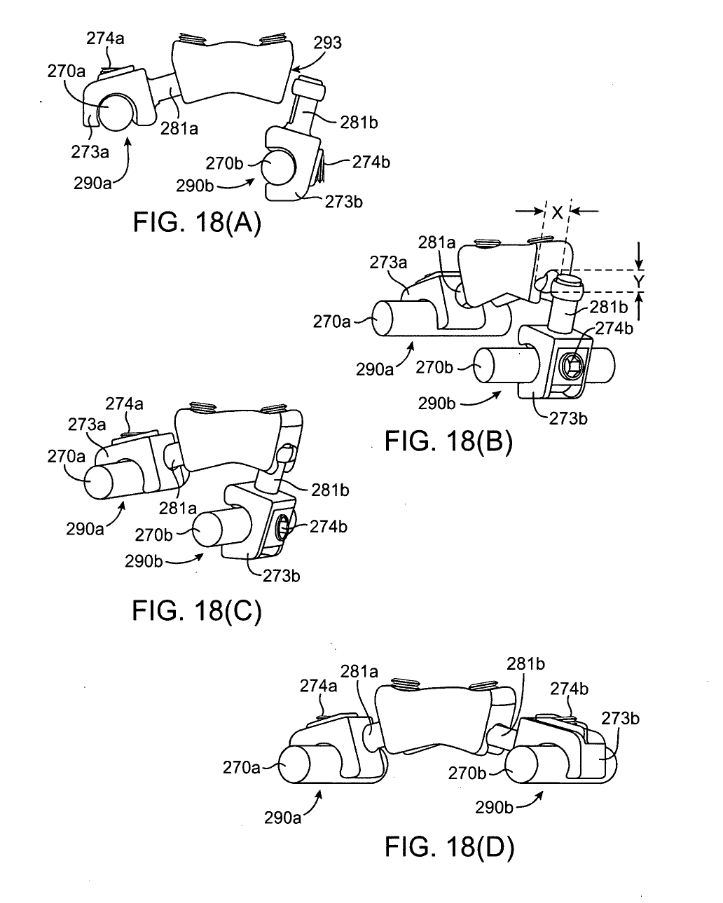

The bar clamping screws 278a and 278b may act directly on the swivels 282a and 282b, and on the bars 281a and 281b, and may serve to frictionally secure the combination against movement following installation. To install the swivels onto the bar, the same may be either slid on or snap-fit over. To install the swivels and bars into the cross connector 280, the swivel, bar, and rod attachment element combination may be rotated to the position shown in FIG. 18(A). The swivel may then be inserted through a swivel insertion slot 293, which as shown in FIG. 18(B) has a horizontal dimension X and a vertical dimension Y. The vertical dimension Y is less than the outer radius of the swivel. The swivel has a substantially spherical surface to accommodate polyaxial orientations prior to tightening of all screws. The housing 276 is provided with a slot on its general underside to accommodate the bar in this position.

FIG. 18(B) shows the swivel partially inserted in the cross connector 280, and FIG. 18(C) shows the swivel fully inserted in the cross connector 280. Following this full insertion, the swivel, bar, and rod attachment element combination may be rotated to the position shown in FIG. 18(D), which is approximately the appropriate position for use in a patient.

In use, after the rods 270a and 270b are attached to the installed pedicle screws, the rod attachment elements may be attached to the rods by placing the rods in the rod-receiving channels 290a and 290b, following the procedures of FIGS. 17(A)-(D), and tightening screws 274a and 274b. The bars 281a and 281b along with their corresponding swivels 282a and 282b may then be disposed in the voids of the cross connector 280 in the manner described by FIGS. 18(A)-(D). The screws 278a and 278b may then be tightened, securing the swivels and bars in the cross connector. In an alternative embodiment, the cross connector is connected to the bars first, and then the rod attachment elements are secured to the rods.

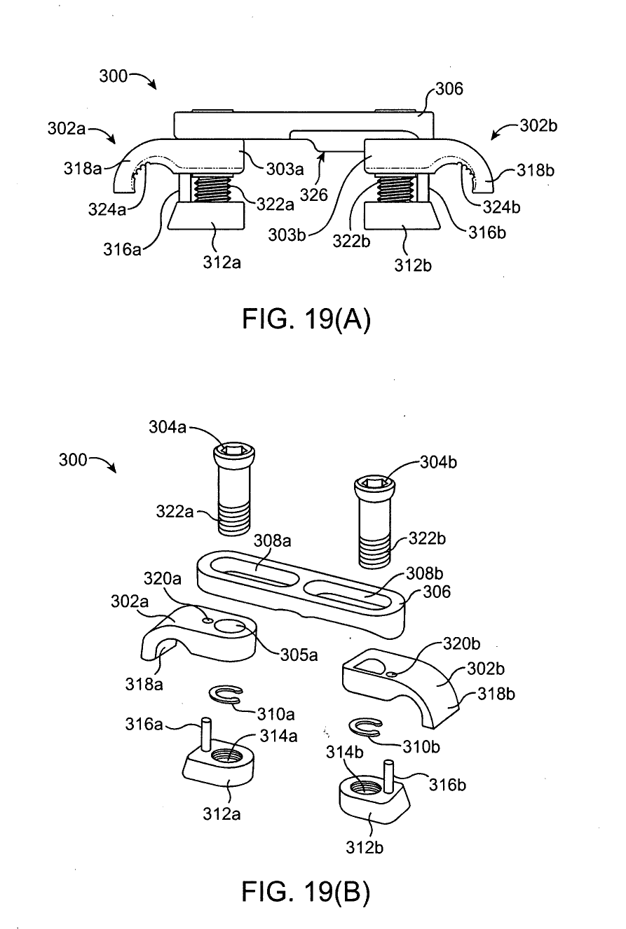

In some embodiments, the bars may be omitted, and the rod attachment elements may attach directly to a cross connector. For example, referring to FIGS. 19(A)-(B) and 20(A)-(C), a cross-connector system 300 is shown with rod attachment elements 302a and 302b, a cross connector 306 spanning them. The rod attachment element 302a includes a housing 303a with a throughhole 305a defined therein, a biasing section or hook section 318a, and a post hole 320a defined therein. The rod attachment element 302b has similar components, although the structure of rod attachment element 302b may be entirely different if dictated by the requirements of the user.

A base 312a is provided corresponding to each rod attachment element 302a, the base 312a including a threaded hole 314a and a post 316a. When constructed, a c-clip 310a is disposed between the rod attachment element housing 303a and the base 312a. The c-clip 310a may snap onto a thread or groove on the screw, so that, in combination with the head on the screw, the cross connector and the rod attachment element are frictionally engaged. The rod attachment element 302b may employ similar components.

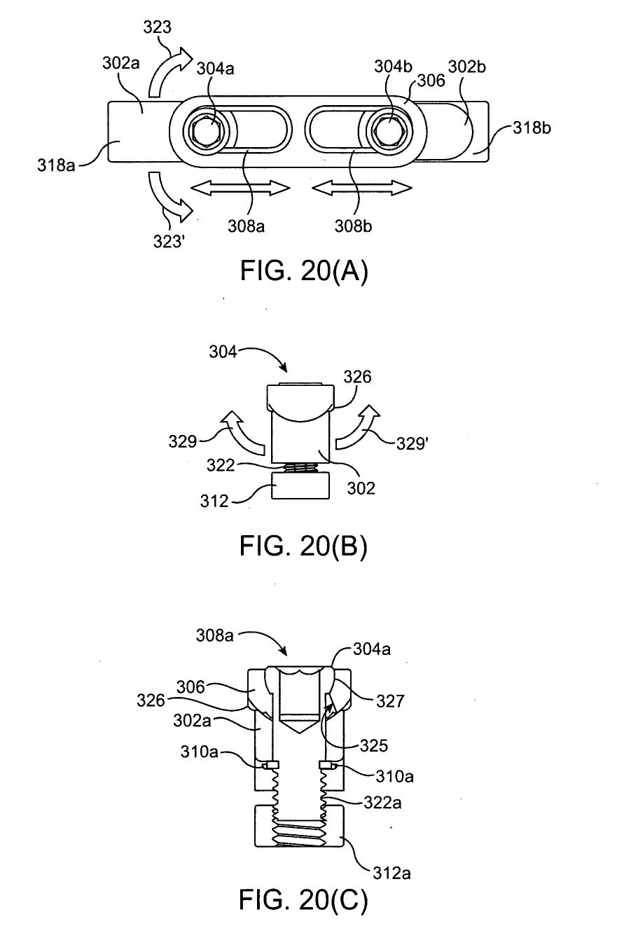

The cross connector 306 has one or more holes defined therein, which are shown in FIG. 19(B) as holes 308a and 308b. The holes may be elongated, as shown, to allow a set of screws 304a and 304b to occupy a variety of locations along the elongated hole, as may be required (see FIG. 20(A)). The holes 308a and 308b may be provided with a depression, so that when a screw is inserted therethrough, the screw head is flush with or below the level of the cross connector 306 (see FIG. 20(C)). The screws 304a and 304b serve to attach the cross connector to the rod attachment elements and also to attach the rods to the rod attachment elements. The hook section 318a includes an interior surface 324a, preferably with a roughened surface. In FIG. 19(A), the interior surface 324a is shown with a number of teeth disposed thereon. Other roughened forms may also be using, including serrations, grit-blasted surfaces, textured coatings and the like. The post 316a engages the post hole 320a so that the base 312a maintains a fixed, e.g., unrotating, position with respect to the housing 303a as the screw 304a is threadingly inserted into the hole 314a in the base 312a.

While the post and post hole maintain the relative positions of the rod attachment element housing and base, the entire rod attachment element may be rotated if desired about the screw 304a. In particular, the longitudinal axis of rod attachment element 302a need not be collinear with the longitudinal axis of rod attachment element 302b. As seen in FIG. 20(A), the rod attachment element 302a may be rotated relative to the cross connector 306, in the angular directions indicated by the arrows 323 and 323′.

The rod attachment elements' pivot, in the directions indicated by the arrows 323 and 323′, may be in part arrested by rounded edges 326 on the underside of the cross connector 306. The rounded edges 326 may be disposed on one or both sides of the cross connector, and at one or two places on each side (to accommodate both directions 323 and 323′). The rounded edges may by configured to gradually increase the stopping force present as the rod attachment elements are pivoted to extreme angles.

As seen in FIG. 20(C), the depression of hole 308a may have a spherical shape 327 on which sits the screw head of screw 304a, and the screw head itself may have a spherical shape. Thus, the screw head and screw may pivot adjacent and with respect to the depression of hole 308a. To accommodate the screw shank movement during pivoting, an underside 325 of the hole 308a may be tapered as shown. Due to these cooperating engaged surfaces, the rod attachment elements may be able to pivot along the directions shown by arrows 329 and 329′, as shown in FIG. 20(B).

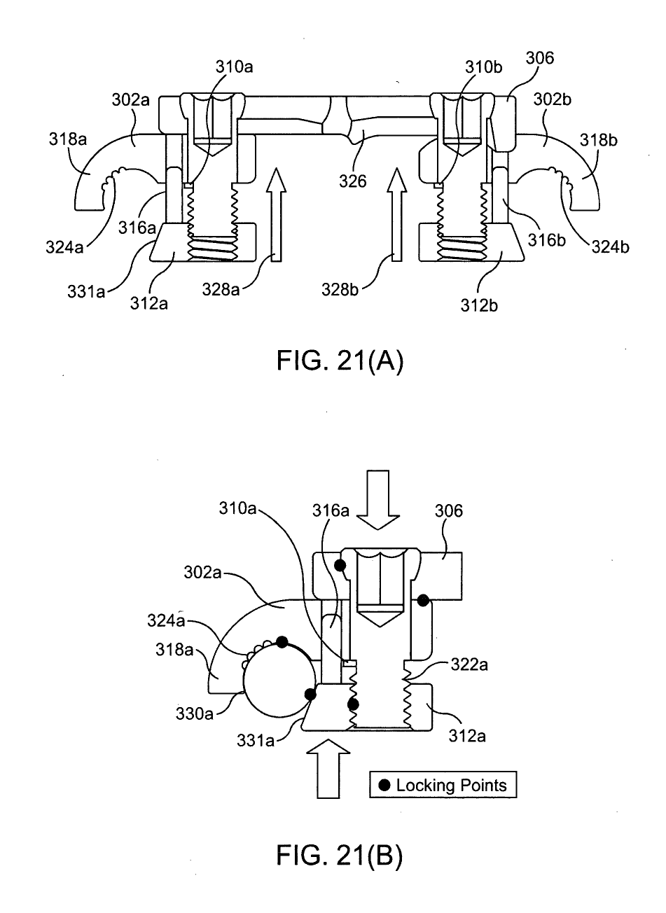

Referring to FIG. 21(A)-(B), threading insertion of the screw 304a causes the base 312a to move in a direction indicated by arrow 328a. Such displacement brings an angled surface 331a of the base 312a into engagement with the rod, securing the same against removal. The rod may be further secured by employment of an undercut 330a which may form a distal end of the hook 318a (see FIG. 21(B)). Contact or locking points for the system are shown in FIG. 21(B) by black dots.

In use, after the rods are attached to the installed pedicle screws, the rod attachment elements may be attached to the rods by first placing the rods in the rod-receiving channels within hook sections 318a and 318b. Then, posts 316a and 316b are placed in post holes 320a and 320b. The screws 304a and 304b are then at least partially tightened, and c-clips 310a and 310b may be disposed on the threads or grooves of screws 304a and 304b. As noted above, the use of posts ensures that angled surfaces 331a and 331b remain directed against the rods, securing the same from movement or removal. As above and in other embodiments, the system geometry, such as fine adjustments of widths and angles between components, may be adjusted prior to final screw tightening.

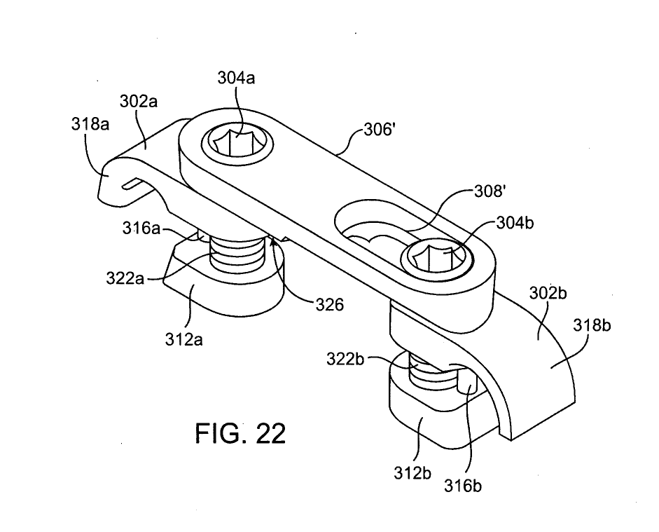

A related embodiment is shown in FIG. 22, in which a cross connector 306′ is shown with a single slot 308′. In this embodiment, the single slot 308′ still allows a degree of width adjustment to achieve a desired distance between rods. The single slot 308′ can of course be provided for attachment to either rod attachment element. The method of use of the embodiment of FIG. 22 is analogous to the method of use of the preceding embodiment, except that the width adjustment is accomplished via only one screw, in particular, screw 304b sliding in slot 308′.

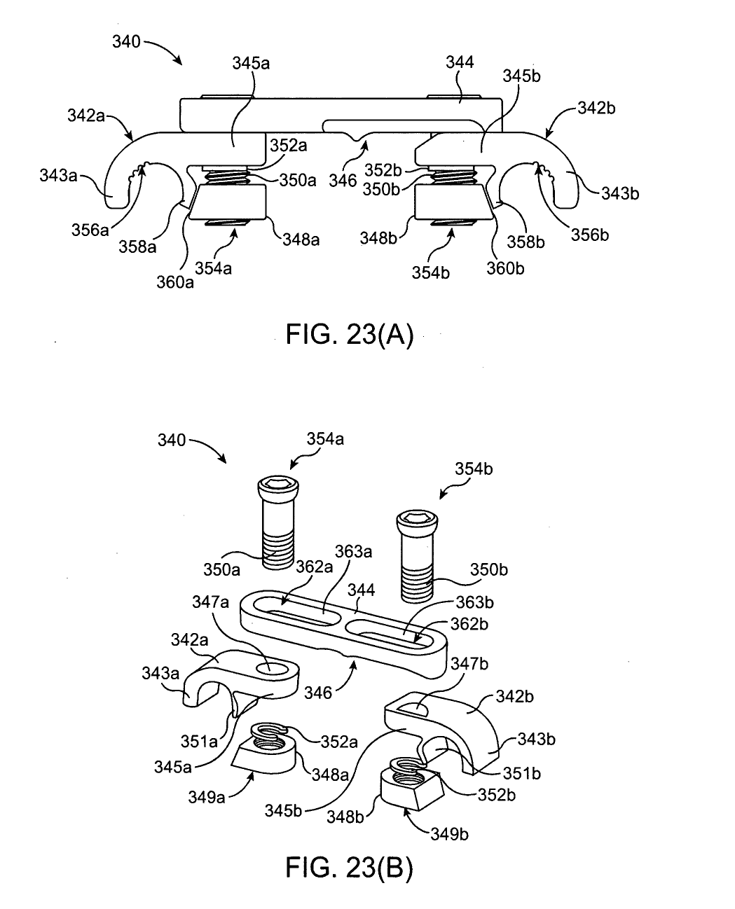

FIGS. 23(A)-(B) show another related embodiment. In this embodied system 340, a cross connector 344 spans two rod attachment elements 342a and 342b. The rod attachment element 342a includes a body 345a with a throughhole 347a defined therein and a biasing section or hook section 343a. The rod attachment element 342b has similar components, although the structure of rod attachment element 342b may be entirely different if dictated by the requirements of the user.

A base 348a is provided corresponding to each rod attachment element 342a, the base 348a including a threaded hole 349a and an angled rod-locking surface 360a. When constructed, a c-clip 352a is disposed between the rod attachment element housing 345a and the top of the base 348a, for the same purpose as is described above. The rod attachment element 342b may employ similar components.

The cross connector 344 has one or more holes defined therein, which are shown in FIG. 23(B) as holes 362a and 362b. The holes may be elongated, as shown, to allow a set of screws 354a and 354b to occupy a variety of locations within the elongated hole, as may be required to accommodate different patient spinal dimensions. The holes 362a and 362b may be provided with depressions 363a and 363b, as seen in FIG. 23(B), so that when a screw is inserted therethrough, the screw head may be made flush with or below the level of the cross connector 344. The screws 354a and 354b serve to attach the cross connector 344 to the rod attachment elements 342a and 342b and also to attach the rods to the rod attachment elements, as will be shown.

The hook section 343a includes an interior surface 356a with a roughened surface. In FIG. 23(A), the interior surface 356a is shown with a number of teeth disposed thereon. Other roughened forms may also be using, including serrations and the like. As may be seen in FIG. 23(A), the rod-contacting surface 356a has a substantially cylindrical cross-section; at an extremal point on the circumference of this cylindrical cross-section, at the end nearest the hole 347a, a flange 358a may downwardly depend, the flange 358a having an angled surface 351a for sliding frictional engagement with an angled surface 360a of the base 348a. Unlike the previous embodiment, no post or post hole is employed in this embodiment; instead, the interaction and engagement between angled surfaces 351a and 360a maintain the fixed, e.g., relatively unrotated, position with respect to the housing and the base as the screw is threadingly inserted into the hole in the base. The screw, in this embodiment as well as others, may incorporate a distal thread section that is deformed or otherwise configured so as to prevent disassembly. The distal thread section or the threaded hole may alternatively or in addition incorporate a locking feature such as a polymer insert or the like.

As in the previously-described embodiment of FIGS. 19-21, the screw head may be made spherical, and the depression appropriately configured as described in connection with those figures, to allow a degree of pivot to accompany this embodiment.

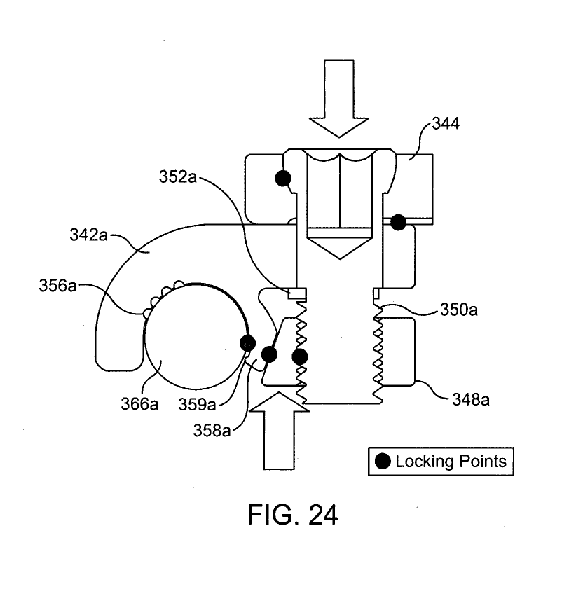

On the side of the flange 358a opposite that of the surface 351a, an undercut 359a may be formed (see FIG. 24), which assists in the securing of the rod to the rod attachment element. The flange may form a flexible hinge, allowing the hook section to be snap-fit around the rod.

As in the previous embodiment, the entire rod attachment element may be rotated if desired about the screw 354a. In particular, the longitudinal axis of rod attachment element 342a need not be collinear with the longitudinal axis of rod attachment element 342b or with the cross connector 344.

In a similar way as noted above in connection with FIGS. 19-20, the rod attachment elements' pivot angle, in the plane parallel to the cross connector 344, may be in part arrested by rounded edges 346 on the underside of the cross connector 344. The rounded edges 346 may be disposed on both sides of the cross connector, and at two places on each side (to accommodate both clockwise and counter-clockwise). The rounded edges may be configured to gradually increase the stopping force present as the rod attachment elements are pivoted to their extreme angles.

Contact or locking points for the system are shown in FIG. 24 by black dots.

In use, after the rods are attached to the installed pedicle screws, the rod attachment elements may be attached to the rods by first placing the rods in the rod-receiving channels within hook sections 343a and 343b. Then base 348a and 348b are threaded onto screws 354a and 354b such that surfaces 351a and 360a, as well as 351b and 360b, are adjacent. The screws 354a and 354b are then tightened, and c-clips 352a and 352b may be disposed on the threads or grooves of screws 354a and 354b. In an alternative embodiment, the cross-connector system may be assembled, or partially assembled, prior to attachment of the rod attachment elements to the rods.

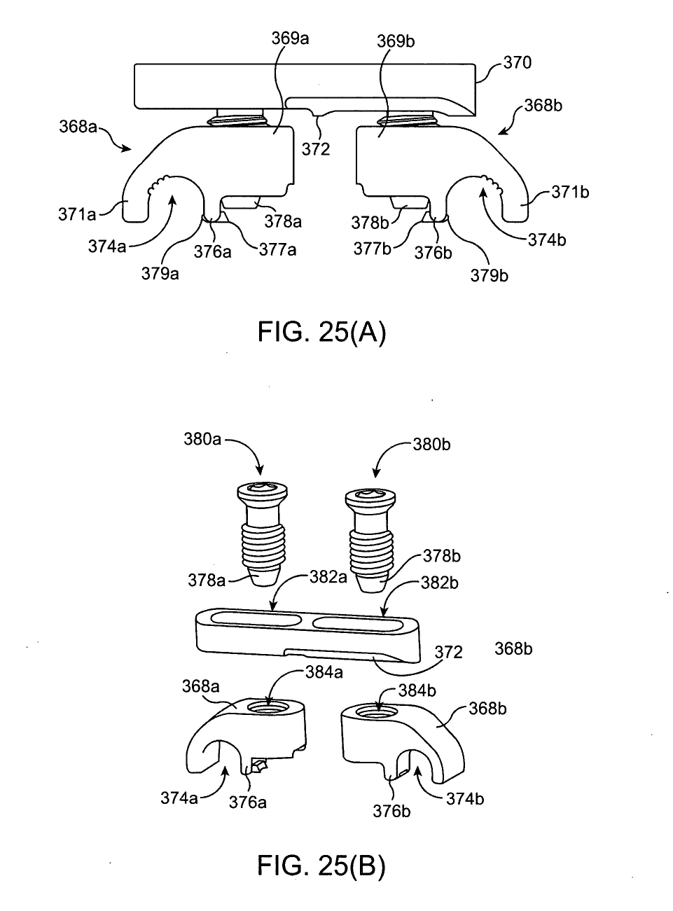

Referring to FIGS. 25(A)-(B), an embodiment is shown with certain similarities to prior-described embodiments, although the rod attachment mechanism is different.

In this embodied system, a cross connector 370 spans two rod attachment elements 368a and 368b. The rod attachment element 368a includes a body 369a with a throughhole 384a defined therein and a biasing section or hook section 371a. The rod attachment element 368b has similar components, although the structure of rod attachment element 368b may be entirely different if dictated by the requirements of the user.

The cross connector 370 has one or more holes defined therein, which are shown in FIG. 25(B) as holes 382a and 382b. The holes may be elongated, as shown, to allow a set of screws 380a and 380b to occupy a variety of locations within the elongated hole, as may be required. The holes 380a and 380b may be provided with a depression, as in the prior embodiments, so that when a screw is inserted therethrough, the screw head may be made flush with or below the level of the cross connector 370. The screws 380a and 380b serve to attach the cross connector 370 to the rod attachment elements 368a and 368b and indirectly also assist in the attachment of the rods to the rod attachment elements, as will be shown.

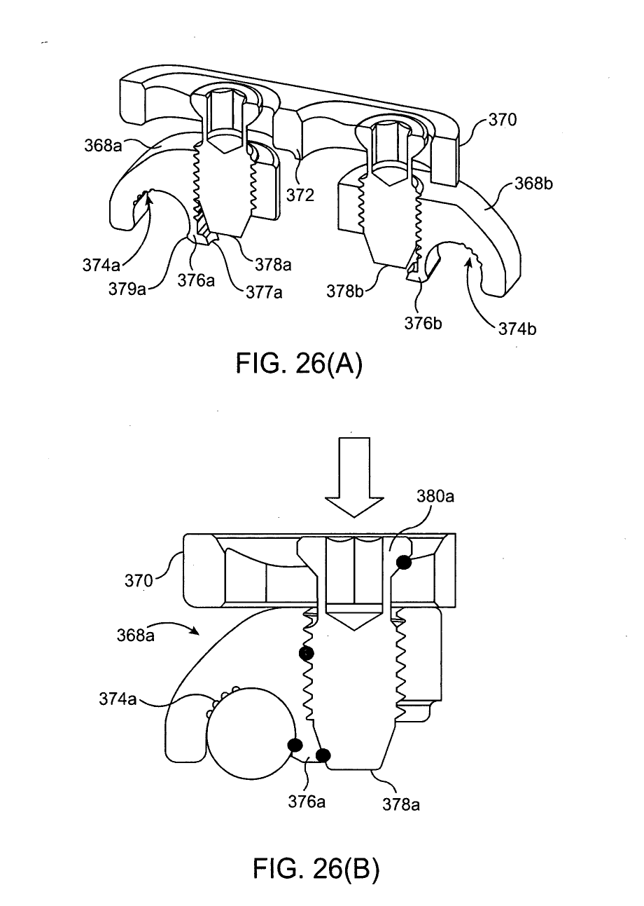

The hook section 371a includes an interior surface 374a with a roughened surface. In FIG. 25(A), the interior surface 374a is shown with a number of teeth disposed thereon. Other roughened forms may also be using, including serrations and the like. As may be seen in FIG. 25(A), the rod-contacting surface 374a has a substantially cylindrical cross-section; at an extremal point on the circumference of this cylindrical cross-section, at the end nearest the hole 384a, a flange 376a may downwardly depend. On the side of the flange 376a opposite the rod-contacting surface, the flange 376a may incorporate a projection 377a. On the side of the flange 376a adjacent the rod-contacting surface, the flange 376a may incorporate an undercut 379a. The undercut 379a assists in securing the rod to the rod attachment element; in particular, the flange may form a flexible hinge, allowing the hook section to be partially snap-fit around the rod. FIG. 26(A) shows a perspective cross-sectional view of this embodiment. The snap-fit is enhanced, or in some cases may be supplanted, by the action of the screw on the flange. In particular, as the screw 380a is inserted through hole 382a and further threadingly inserted into hole 384a, a distal end 378a of the screw 380a contacts the projection 377a and forces the same towards the rod. As the projection 377a is forced in that direction, so is the undercut 379a, and the undercut 379a further contacts and surrounds the rod, and secures the same against removal.

No base need be employed in this embodiment. As in the previously-described embodiments, the screw head may be made spherical, and the depression appropriately configured as described in connection with those figures, to allow a degree of pivot (prior to final screw tightening) to accompany this embodiment.

In a similar way as noted above in connection with FIGS. 19-20 and 23, the rod attachment elements' pivot angle, in the plane parallel to the cross connector 370, may be in part arrested by rounded edges 372 on the underside of the cross connector 370. The rounded edges 372 may be disposed on both sides of the cross connector, and at two places on each side (to accommodate both clockwise and counter-clockwise). The rounded edges may be configured to gradually increase the stopping force present as the rod attachment elements are pivoted to their extremal angles.

Contact or locking points for the system are shown in FIG. 26(B) by black dots.

In use, after the rods are attached to the installed pedicle screws, the rod attachment elements may be attached to the rods by first placing the rods in the rod-receiving channels within hook sections 371a and 371b. In many procedures, for this embodiment and for the others, a rod attachment element is attached to one rod, and the system is partially assembled. The width between the cross-connector is then accommodated by modification of the cross-connector and/or one or more rod attachment elements. A rod attachment element is secured to the second rod, and a final screw tightening may then occur. Then screws 380a and 380b are installed such that their distal ends deflect projection 379 and undercut 377 such that the undercut is forced against the rod, securing the same against movement.

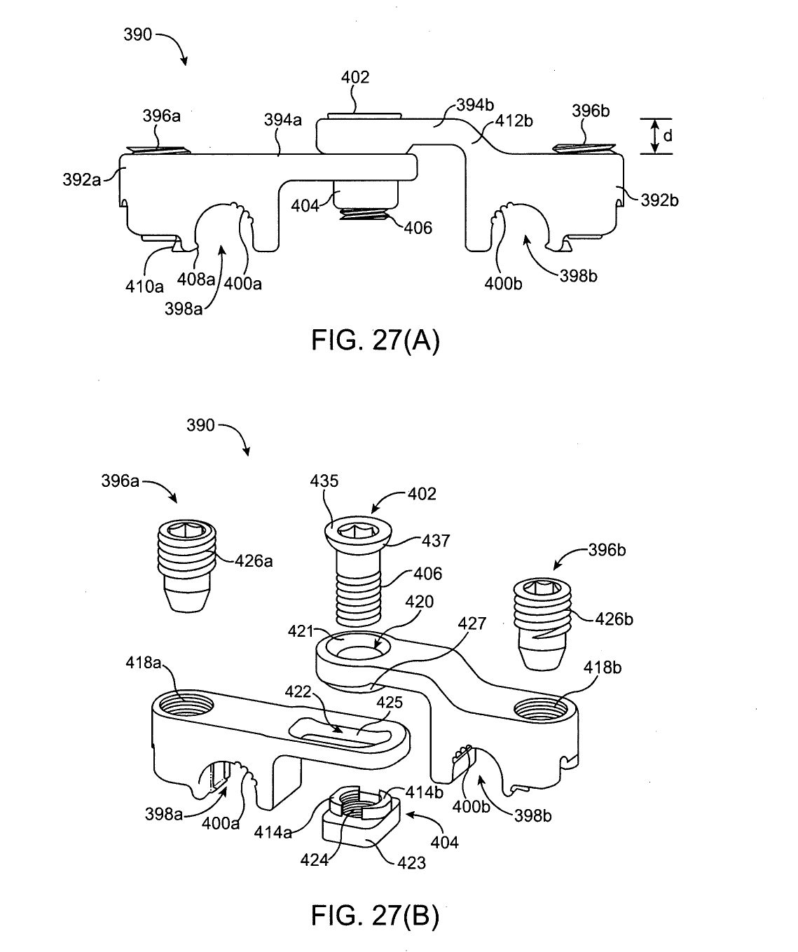

Another embodiment of the invention is shown in FIGS. 27-30. Referring in particular to FIGS. 27(A)-(B), a system 390 is shown with two rod attachment elements 392a and 392b. Within each is defined a substantially cylindrical opening 398a and 398b for receipt and securing of a rod. The cylindrical openings may further include a serrated portion 400a and 400b with the same purpose as above, to assist in the securing of a rod. The rod attachment elements 392a and 392b further incorporate threaded holes 418a and 418b for threading insertion of rod-locking screws 396a and 396b. The rod-locking screws 396a and 396b have threads 426a and 426b disposed thereon. The rod attachment elements 392a and 392b further incorporate first and second bars 394a and 394b. The bar 394a may extend directly out from the rod attachment element 392a, while the bar 394b may be vertically displaced a distance d from rod attachment element 392b via diagonal section 412b. Of course, in an alternative embodiment, rod attachment element 392a may incorporate the vertically-displaced bar, or both may have vertically-displaced bars, where the amount of vertical displacement differs and/or is in opposite directions.

Each bar may have a through-hole defined therein. In FIG. 27(B), the bar 394a has through-hole 422, and the bar 394b has through-hole 420. One or both through-holes may be elongated to accommodate a range of widths between the rods. In FIG. 27(B), the through-hole 422 is shown elongated.

A bar clamping screw 402 having threads 406 holds the bars in a secure fashion via a nut 404. The nut 404 includes a base section 423 through which is defined a threaded hole 424. Depending upwardly from the base section 423 are two projections 414a and 414b. The projections 414a and 414b, which may vary in number, are received within the elongated hole 422 of the bar 394a and serve to prevent the nut 404 from turning when the screw 402 is threadingly inserted.

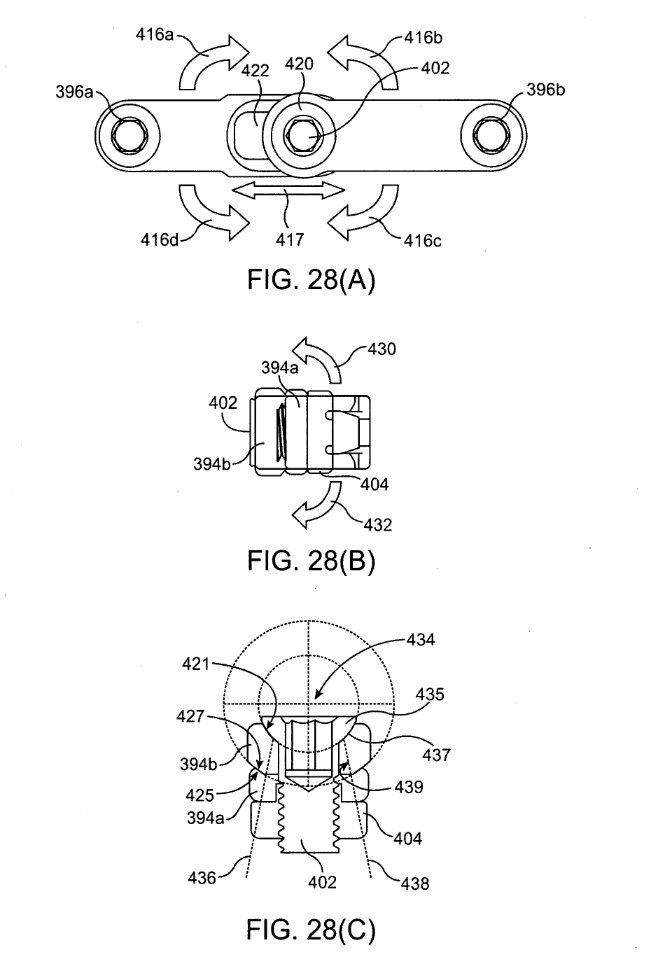

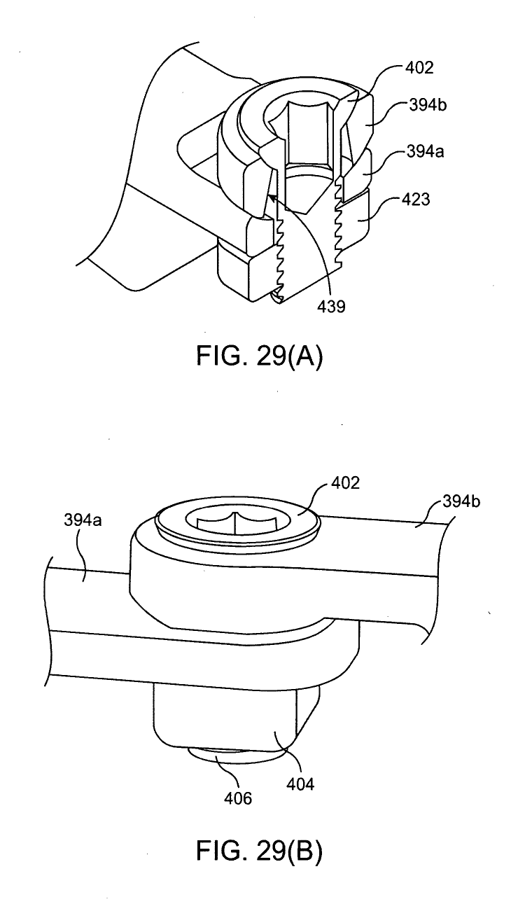

The construction as described above allows a number of degrees of freedom to be obtained by the system 390 (these degrees of freedom may all be removed by final screw tightening—or one or more may remain “free”—such as to allow a degree of motion after implantation). FIG. 28(A) shows arrows 416a-416d, which indicate a degree of rotational freedom about an axis defined by the longitudinal axis of the screw 402. An arrow 417 indicates a degree of translational freedom due to the elongated hole 422. FIG. 28(B) shows another rotational degree of freedom enjoyed by the system 390, this degree of freedom transverse to the plane defined by the bars. The degree of freedom indicated by arrows 430 and 432 is afforded by the construction of the system 390 as indicated in FIG. 28(C) and FIGS. 29(A)-(B).

As seen in FIG. 28(C) and FIG. 27(B), a bottom surface 437 of a head 435 of screw 402 may be constructed to be substantially spherical, and the same may rotationally engage a spherical taper 421 at the top of the hole 420. In the same way, the bottom of the hole 420 may be provided with a spherical taper 427, and the same may rotationally engage a spherical taper 425 at the top of the hole 422. The hole 420 may itself incorporate a wall 439 having a taper as indicated by lines 436 and 438 of FIG. 28(C).

In other words, to further assist in providing the degree of freedom indicated by arrows 430 and 432, the bottom of the bar 394b, and the top of the bar 394a, may be provided with mating spherical tapers so that one may be slidingly rotated on top of the other, as indicated in FIGS. 28(C) and 29(A). In many procedures, the degrees of freedom may be used to orient a device in a proper position and then the screw 402 may be threadingly tightened, locking the system in that position, as shown in FIG. 29(B).

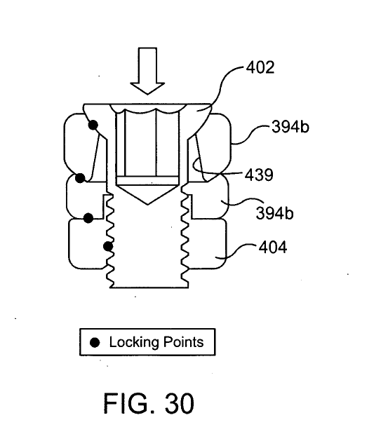

Contact or locking points for the system are shown in FIG. 30 by black dots.

In use, after the rods are attached to the installed pedicle screws, the rod attachment elements may be attached to the rods by first placing the rods in the rod-receiving channels defined by surfaces 398a and 398b. Then screws 396a and 396b are installed such that their distal ends deflect projection 410 and undercut 408 such that the undercut is forced against the rod, securing the same against movement. The bars are then positioned such that the screw 402 may extend through the hole in each. The nut 404 is then positioned such that the screw 402 may be threadingly inserted into the same, with the projections 414a and 414b inserted into the hole 422 to arrest rotational movement of the nut. Of course, in an alternative embodiment, the bars may be secured together first, and the same later attached to the rods.

In all of the above-described embodiments, where descriptions are provided for a group of elements suffixed by the letter ‘a’, a similar description may apply for the group of elements suffixed by the letter ‘b’; however, in all cases, a different type of group of elements may also be employed. There is no requirement that the same elements be employed. For example, a cross-connector system may employ two different rod attachment elements of entirely different type, if dictated by the requirements of the user. Whether the rod attachment elements are of the same or of differing types, the way in which the same couple to the rods may differ. One may couple at a different angle than the other. One may couple in a dynamic way, while the other couples in a static way. One may couple in a reversible fashion, while the other couple irreversibly. They may attach to different size rods, including rods of different lengths or diameters or both. The materials of construction of the rod attachment elements may differ. The bars may attach to the rod attachment elements at virtually any angle, and as noted may be pre-attached or integral therewith.

Further, in all of the above-described embodiments, various types of locking screws may be employed to protect against disassembly. Such locking screws may include polymer inserts, deformed or other high-resistance threads, or other types of locking mechanisms. The heads of the screws may incorporate insertable or removable fills or inserts so as to prevent contamination from entering a portion of the head where engagement with a tool may occur. In this way, follow-up adjustments and removal procedures may be made more convenient.

The rods described above may be of the type disclosed in U.S. patent application Ser. No. 11/362,366, filed on Feb. 23, 2006, entitled “Systems and methods for stabilization of bone structures” and incorporated by reference in its entirety herein.

The materials used in construction of all of the components are typically biocompatible and may be metal, such as titanium, although rigid plastics may also be employed.

Components disclosed above may be employed in various combinations.

Each rod attachment element may further include a hydraulic or pneumatic component, e.g., a hydraulic assembly that compresses the clamp portion to grip a corresponding rod. Other devices conveying a mechanical advantage to improve the gripping force may also be employed, such as cams, gear assemblies, and the like.

While the invention has been described in the context of spinal fusion, the same may be employed in dynamic systems, and indeed may include dynamic elements either in the cross-connector or as parts of the stabilization rods to which the rod attachment elements connect. Embodiments of the invention may also be employed in various other systems, such as facet replacement or facet augmentation systems.

We claim:

1. A spinal stabilization system for a patient, comprising:
a. A first rod attachment element to attach to a first vertebral stabilization rod;
b. A second rod attachment element to attach to a second vertebral stabilization rod;
c. A first bar to attach to the first rod attachment element;
d. A second bar to attach to the second rod attachment element; and
e. A connector configured to connect the first and second bars;
f. Wherein at least one rod attachment element has a two-part design, wherein one part of the two-part design contacts one portion of a corresponding rod and the other part of the two-part design contacts an other portion of the corresponding rod to capture the rod.

2. The system of claim 1, wherein the at least one rod attachment element with a two-part design is configured as a hinged hook and pivoting clamp design, as a two-piece scissor design, or as a hook and sliding clamp design.

3. The system of claim 1, wherein the at least one rod attachment element with a two-part design is configured with two parts having a common rod-receiving channel, wherein the two parts are rotatable with respect to each other about the common rod-receiving channel, and wherein at one rotational position a rod may be received in the common rod-receiving channel, and wherein at another rotational position the rod may be captured in the common rod-receiving channel.

4. The system of claim 2, wherein the first or second rod attachment element clamps to its respective vertebral stabilization rod upon insertion of a screw.

5. The system of claim 1, wherein an end of at least one of the first or second bar opposite the first or second rod attachment element, respectively, includes a polyaxial swivel.

6. The system of claim 1, wherein at least one of the first bar, the second bar, or the connector includes a dynamic element.

7. The system of claim 1, wherein the first or second bar clamps to the connector upon insertion of a screw.

8. The system of claim 7, wherein both the first and second bars clamp to the connector upon insertion of a screw.

9. The system of claim 8, wherein the connector has at least one elongated hole for receipt of the screw.

10. The system of claim 8, wherein the configuration of the first and second bars and the connector allows the first bar to rotate relative to the second bar.

11. The system of claim 1, wherein the first and second bars are configured to engagedly mate with each other.

12. The system of claim 1, wherein at least one of the first or second rod attachment element further comprises a rod-contacting surface, wherein the rod-contacting surface includes a ridged surface.

13. The system of claim 1, wherein the connector comprises:
a. An upper housing;
b. A lower housing; and
c. A screw that secures the upper and lower housings,
d. Wherein the connector further comprises at least one void for receipt of a bar.

14. The system of claim 5, wherein the connector comprises:
a. An upper housing;
b. A lower housing; and
c. A screw that secures the upper and lower housings,
d. Wherein the connector further comprises at least one void for receipt of a swivel.

15. The system of claim 1, wherein the connector comprises a component having a throughhole to receive the first and second bars in an overlapping configuration, wherein the component further comprises a threaded hole to receive a screw.

16. The system of claim 11, wherein one of the first and second bars includes a nub, and the other of the first and second bars includes a corresponding recess, wherein the nub engages the recess to hold the first and second bars together at least one point.

17. The system of claim 15, wherein the first and second bars overlap within the cross connector, and wherein within the overlap area the first and second bars include cooperating engaged surfaces, such that the first and second bars are rotatable relative to each other.

18. The system of claim 15, wherein the upper and lower housing form an entrance hole, and wherein the swivel may be received in the entrance hole in one orientation, and wherein the swivel may be captured in the connector in another orientation.

19. The system of claim 5, wherein at least one of the first or second bars includes a groove on which the swivel is slidingly disposed.

20. A method for stabilizing a patient's spine, comprising:
a. Installing a first set of two pedicle screw systems into a superior vertebral segment;
b. Installing a second set of two pedicle screw systems into an inferior vertebral segment;
c. Connecting a first rod between one of the pedicle screw systems in the first set and one of the pedicle screw systems in the second set;
d. Connecting a second rod between the other of the pedicle screw systems in the first set and the other of the pedicle screw systems in the second set;
e. Connecting a first rod attachment element to the first rod;
f. Connecting a second rod attachment element to the second rod;
g. Connecting a first bar to the first rod attachment element;
h. Connecting a second bar to the second rod attachment element;
i. Connecting a cross connector to both the first bar and the second bar;
j. Wherein at least one rod attachment element has a two-part design, wherein one part of the two-part design contacts one portion of a corresponding rod and the other part of the two-part design contacts an other portion of the corresponding rod to capture the rod.

21. The method of claim 20, wherein the at least one rod attachment element with a two-part design is configured as a hinged hook and pivoting clamp design, as a two-piece scissor design, or as a hook and sliding clamp design.

22. The method of claim 20, wherein connecting a first or second rod attachment element further comprises threadingly inserting a screw into the first or second rod attachment element.

23. The method of claim 22, further comprising threadingly inserting the screw such that a distal end of the screw flexes a projection and an undercut and such that the undercut contacts and frictionally secures a rod.

24. The method of claim 20, wherein connecting a first or second rod attachment element further comprises inserting a sliding clamp into a hook and tightening a screw into the rod attachment element, such that the screw frictionally engages the sliding clamp.

25. The method of claim 20, wherein connecting a first or second rod attachment element further comprises inserting a rod into a rod-receiving channel and rotating a first portion of the rod attachment element relative to a second portion of the rod attachment element, such that the rod is locked within the rod-receiving channel.

26. The method of claim 20, wherein connecting a cross connector to the first or second bar further comprises moving the bar to a first orientation, inserting an end of the bar into the cross connector, and moving the bar to a second orientation.

27. The method of claim 20 wherein either the first or second rod or both include a pre-attached or integral rod attachment element.

28. The method of claim 20 wherein at least one of the first or second rod attachment elements include an integral or pre-attached first bar.

29. A spinal stabilization system for a patient, comprising:
a. A first rod attachment element to attach to a first vertebral stabilization rod;
b. A second rod attachment element to attach to a second vertebral stabilization rod; and
c. A cross-connector configured to connect the first and second rod attachment elements;
d. Wherein at least one rod attachment element has a two-part design, wherein one part of the two-part design contacts one portion of a corresponding rod and the other part of the two-part design contacts an other portion of the corresponding rod to capture the rod.

30. A method for stabilizing a patient's spine, comprising:
a. Installing a first set of two pedicle screw systems into a superior vertebral segment;
b. Installing a second set of two pedicle screw systems into an inferior vertebral segment;
c. Connecting a first rod between one of the pedicle screw systems in the first set and one of the pedicle screw systems in the second set;
d. Connecting a second rod between the other of the pedicle screw systems in the first set and the other of the pedicle screw systems in the second set;
e. Connecting a first rod attachment element to the first rod;
f. Connecting a second rod attachment element to the second rod; and
g. Connecting a cross-connector between the first and second rod attachment elements;
h. Wherein at least one rod attachment element has a two-part design, wherein one part of the two-part design contacts one portion of a corresponding rod and the other part of the two-part design contacts an other portion of the corresponding rod.

31. The method of claim 30, wherein at least one rod attachment element with a two-part design is configured as a hinged hook and pivoting clamp design, as a two-piece scissor design, or as a hook and sliding clamp design.

32. The method of claim 30, wherein either the first or second rod or both include a pre-attached or integral rod attachment element.

33. The method of claim 30, wherein one of the first or second rod attachment elements include an integral or pre-attached cross-connector.

34. A spinal stabilization system for a patient, comprising:
a. A first rod attachment element to attach to a first vertebral stabilization rod, the first rod attachment element including a first biasing section;
b. A second rod attachment element to attach to a second vertebral stabilization rod, the second rod attachment element including a second biasing section;
c. A first bar to attach to the first rod attachment element;
d. A second bar to attach to the second rod attachment element; and
e. A connector configured to connect the first and second bars;
f. Wherein at least the first rod attachment element further comprises a rod-contacting surface and a corresponding screw having a head with a cam section, wherein rotation of the screw having a head with a cam section into the rod attachment element forces the cam section towards the rod-contacting surface, capturing the rod between the rod-contacting surface and the cam section.

35. The system of claim 34, wherein either or both of the first or second biasing sections includes a hook section.
